(19) United States
(12) Patent Application Publication
(10) Pub. No.: US 2024/0423980 A1
(43) Pub. Date: Dec. 26, 2024

(54) METHODS OF PREVENTING A VENOUS THROMBOEMBOLIC EVENT

(71) Applicant: Board of Supervisors of Louisiana State University and Agricultural and Mechanical College, Baton Rouge, LA (US)

(72) Inventors: Nirav Dhanesha, Bossier City, LA (US); Nilesh Pandey, Shreveport, LA (US); Harpreet Kaur, Shreveport, LA (US)

(21) Appl. No.: 18/750,975

(22) Filed: Jun. 21, 2024

Related U.S. Application Data
(60) Provisional application No. 63/522,823, filed on Jun. 23, 2023.

(51) Int. Cl. A61K 31/506; A61K 31/37; A61K 31/4439; A61K 31/4545; A61K 31/5377; A61K 31/727; A61K 45/06; A61P 7/02

(52) U.S. Cl. CPC A61K 31/506; A61K 31/37; A61K 31/4439; A61K 31/4545; A61K 31/5377; A61K 31/727; A61K 45/06; A61P 7/02

(57) ABSTRACT

This invention is directed to methods of preventing, treating, or alleviating a symptom of a venous thromboembolic event in a subject.

This application claims priority from U.S. provisional patent application No. 63/522,823, filed on Jun. 23, 2023, the entire contents of which are incorporated herein by reference.

### GOVERNMENT INTERESTS

This invention was made with government support under R01 HL158546 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to methods of preventing, treating, or alleviating a symptom of a venous thromboembolic event.

### BACKGROUND OF THE INVENTION

Venous thromboembolic (VTE) events, including deep vein thrombosis (DVT) and pulmonary embolism (PE), occur in approximately 2 million Americans per year of which 10-30% of patients die within 30 days. The incidence of VTE increases sharply with age. Young individuals have very low VTE risk, but this risk increases to approximately 1% per year in the elderly, indicating that aging is one of the strongest and most prevalent risk factors for VTE. Similarly, patients with stroke are at a risk for developing VTE events, which can result in a worse clinical outcome and is estimated to affect approximately 80,000 stroke patients each year in the United States. The overall prevalence of clinically evident DVT after acute stroke is approximately 10 percent. The prevalence of asymptomatic DVT is even higher.

### SUMMARY OF THE INVENTION

The present invention provides methods of preventing, treating, or alleviating a symptom of a venous thromboembolic event in a subject. In embodiments, the method comprises administering to a subject in need thereof an endothelin receptor inhibitor.

In embodiments, the endothelin receptor inhibitor can comprise an integrin alpha9 (ITGA9) inhibitor. In embodiments, the ITGA9 inhibitor can comprise a small molecule, an antibody or fragment thereof, or a nucleic acid. For example, the small molecule can comprise a structure according to the following, or a derivative or analog thereof:

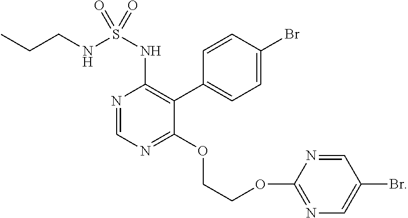 [SMILES: CCCNS(=O)(=O)Nc1ncnc(OCCOc2ncc(Br)cn2)c1-c1ccc(Br)cc1]

In embodiments, the venous thromboembolic event can comprise venous thrombosis, pulmonary embolism, or cancer-associated thrombosis. For example, venous thrombosis can comprise deep vein thrombosis.

In embodiments, the subject is afflicted with a cancer. In embodiments, the cancer can comprise a solid tumor cancer. For example, the solid tumor cancer can comprise lung cancer, brain cancer, stomach cancer, or pancreatic cancer.

In embodiments, the subject is or has been administered a therapeutic regimen associated with increased incidence of a venous thromboembolic event. For example, the additional therapeutic regimen can comprise an anti-cancer therapeutic regimen. For example, the anti-cancer therapeutic regimen can comprise chemotherapy, radiotherapy, immunotherapy, surgery, or any combination thereof.

In embodiments, aspects of the invention can further comprise administering to the subject a thromboprophylaxis regimen. In embodiments, the thromboprophylaxis regimen can comprise an antithrombotic agent. For example, the antithrombotic agent can comprise warfarin, heparin, dabigatran, rivaroxaban, or apixaban.

Other objects and advantages of this invention will become readily apparent from the ensuing description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 provides a schematic showing the docked pose of Macitentan in α9β1 with Macitentan.

FIG. 2 provides a graph showing the inhibition of ITGA9-VCAM1 binding by Macitentan in a cell-free assay.

FIG. 3 provides a graph showing the in vivo effect of Macitentan in a mouse model of deep vein thrombosis (DVT).

FIG. 4 provides a graph showing increased venous thromboembolic event (VTE) risk in stroke patients.

FIG. 5 shows increased DVT incidence in wild type (WT) mice with stroke. Panel A provides a schematic of the experiment. Panel B provides a graph showing thrombosis incidence on day 1 following stroke. Panel C (left) provides a representation of inferior vena cava (IVC) thrombus on day-1 following stroke. Panel C (right) provides a bar graph showing thrombus weight.

FIG. 6 shows neutrophils exhibit increased integrin α9 expression after stroke in humans. Panel A provides a graph showing the quantification of α9 expression in peripheral neutrophils by real-time PCR. Panel B provides a graph showing cytokines and myeloperoxidase levels in neutrophils isolated from each group as analyzed by ELISA.

FIG. 7 shows characterization of the neutrophil specific α9−/− mice. Panel A shows western blot analysis of integrin α9 expression on neutrophils isolated from bone marrow of α9fl/fl and α9fl/flMrp8Cre+/− mice. Panel B provides a graph showing blood neutrophil count from both genotypes. Panel C provides a graph showing tail bleeding time.

FIG. 8 provides data showing neutrophil-specific α9−/− mice were less susceptible to DVT. Panel A provides a graph showing DVT incidence rate. Panel B (left) provides representative images of IVC thrombus from each genotype harvested after 48 hours of IVC stenosis. Panel B (right) provides a graph showing thrombus weight. (Only mice with thrombus included in Panel B). Side branches were not ligated.

FIG. 9 provides representative blots and analysis of pFAK and pSrc, after 30 minutes of PMA (20 ng/mL) stimulation from isolated neutrophils from each group of mice.

FIG. 10 provides data relating to neutrophil specific integrin alpha-9 deficient mice.

FIG. 11 provides data showing increased α9 expression in neutrophils six hours post DVT in an IVC stenosis mouse model.

FIG. 12 provides data showing differentially regulated genes in neutrophils of control and mice with DVT.

FIG. 13 provides data showing that neutrophil-specific α9^{-/-} mice exhibit reduced DVT severity is provided. DVT outcome was evaluated using IVC stenosis in neutrophil-specific α9^{-/-} mice. Significantly reduced DVT incidence and thrombus weight in neutrophil-specific α9^{-/-} mice was observed.

FIG. 14 provides data showing that myeloid cell-specific α9 mice exhibit reduced DVT severity in IVC stasis (Panel A) and stenosis (Panel B) models.

FIG. 15 provides data relating to α9-VCAM1 interactions.

FIG. 16 provides a flow chart showing homology model building is provided.

Integrin α9 Sequence was retrieved from uniport (ID: Q13797). Integrin α4 (PDB ID: 3V4P, α4β1-hetrodimer) unit was used as template protein for model building. β1 subunit (from α4β1-hetrodimer) was merged with the α9 integrin, to form α9β1. Ca2+ and Mg2+ were retained from the α4β1 complex, as they impart stability to the protein and help in the ligand binding. The built model was energetically optimized to remove the steric clashes and was validated.

FIG. 17 provides a flow chart showing the docking steps.

FIG. 18 provides a bar graph showing % inhibition of alpha-9 VCAM-1 binding by Macitentan.

FIG. 19 provides data showing in vivo efficacy of Macitentan in WT mice.

FIG. 20 provides data showing significant expression of neutrophil integrin α9 levels in histopathologically diagnosed non-small cell lung cancer cases. Neutrophils (CD66b+; green); Integrin α9 (ITGA9; red); (n=8).

FIG. 21 provides a schematic showing an optimized murine model of lung cancer which exhibits enhanced coagulopathy is shown. Male WT mice were injected with LLC1 cells, IV or SC. The markers of coagulations were increased in mice with IV injection of LLC1 cells.

FIG. 22 provides data showing neutrophil specific α9 deficient mice exhibit significantly reduced DVT severity in mice with lung-cancer is provided. (Panel A) Left, representative IVC thrombus harvested 48-hour post-stenosis from each group. Right, thrombus weight (mg). Each dot represents a single mouse. (Panel B) Left, representative cross-sectional immunofluorescence image of the isolated IVC thrombus (48-hour post-stenosis) from each group for Ly6G (neutrophils, green), CitH3 (red), and DAPI (blue). Magnification 20×; Scale bar 50 μm. Right, quantification.

FIG. 23 provides data showing that Macitentan treatment significantly reduced DVT severity in mice with lung cancer is provided. Panel A provides a schematic showing the experimental design. Panel B provides data showing representative IVC thrombus harvested 48-hour post-stenosis from each group (Left), and thrombus weight (mg) (Right). Each dot represents a single mouse.

### DETAILED DESCRIPTION OF THE INVENTION

Aspects of the invention are drawn to methods of preventing, treating, or alleviating a symptom of a venous thromboembolic event in a subject.

Detailed descriptions of one or more embodiments are provided herein. It is to be understood, however, that the invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The term “in vivo” can refer to an event that takes place in a subject's body.

The term “in vitro” can refer to an event that takes places outside of a subject's body.

The term “ex vivo” can refer to outside a living subject. Examples of ex vivo cell populations include in vitro cell cultures and biological samples such as fluid or tissue samples from humans or animals. Such samples can be obtained by methods well known in the art. Exemplary biological fluid samples include blood, cerebrospinal fluid, urine, saliva. Exemplary tissue samples include tumors and biopsies thereof.

The term “antagonist” can refer to a compound or composition that can decrease, block, inhibit, abrogate, or interfere with a biological response by binding to or blocking a cellular constituent.

The term “agonist” can refer to a compound or composition that interacts with a cellular constituent and elicits an observable response. For example, an agonist can stimulate an activity at a receptor or receptors normally stimulated by naturally occurring substances, thus triggering a response.

Aspects of the invention are drawn towards methods of preventing, treating, or alleviating a symptom of a venous thromboembolic event in a subject. For example, embodiments as described herein comprise administering to a subject in need thereof an endothelin receptor inhibitor, thereby preventing, treating, or alleviating a symptom of a venous thromboembolic event in the subject.

“Thrombosis” can refer to the formation of thrombus or clot inside a blood vessel resulting in obstruction of blood flow through the circulatory system. The term “venous thromboembolic event” or “venous thromboembolism” can refer to a condition that occurs when a blood clot forms in a vein. Venous thromboembolic events can include deep vein thrombosis (DVT) and pulmonary embolism (PE). DVT can occur when a blood clot forms in a deep vein, such as in the lower leg, thigh, or pelvis. Pulmonary embolism can occur when a clot breaks loose and travels through the bloodstream to the lungs.

In accordance with embodiments of the invention, a subject in need thereof is administered an endothelin receptor inhibitor or a pharmaceutical composition comprising an endothelin receptor inhibitor. Endothelin receptor inhibitors can prevent or treat thrombosis, thrombus formation, thrombotic events and thrombosis-related complications associated with cancer and cancer chemotherapy, or prevent or alleviate symptoms thereof.

The term “inhibitor” (e.g., an endothelin receptor inhibitor or an ITGA9 inhibitor) can refer to a substance having an inhibitory activity against the function of a target molecule such as a compound, an antibody, an anti-sense oligonucleotide (“Antisense Drug Technology: Principles, Strategies, and Applications (Second Edition)”, CRC Press, 2007), an RNAi oligonucleotide (“RNA Methodologies (Third Edition)”, Elsevier, 2005, Chapter 24), a peptide nucleic acid (Kaihatsu et al., Chemistry & Biology, 2004, 11 (6), p. 749-758) and a peptidic antagonist (Ladner et al., Drug Discovery Today, 2004, 9, p. 525-529). Accordingly, inhibitors can encompass numerous classes of chemical molecules, e.g., small organic or inorganic molecules, polysaccharides, biological macromolecules, e.g., peptides, proteins, peptide analogs and derivatives, peptidomimetics, antibodies, antibody fragments, nucleic acids, nucleic acid analogs and derivatives, an extract made from biological materials such as bacteria, plants, fungi, or animal cells or tissues, naturally occurring or synthetic compositions.

The term “endothelin receptor inhibitor” can refer to any agent that blocks and/or inhibits the binding of endothelin to an endothelin receptor, and/or any agent that blocks or inhibits endothelin receptor-mediated signal transduction. For example, the endothelin receptor inhibitor can comprise an integrin alpha9 (ITGA9) inhibitor.

“ITGA9” is one of the integrin subunits that facilitates accelerated cell migration and regulates diverse biological functions such as angiogenesis, lymph angiogenesis, cancer cell proliferation and migration. ITGA9 subunit interacts with beta1 subunit generating α9β1 heterodimer, which is expressed in many cell types such as epithelial cells, neutrophils, hepatocytes, muscle and endothelial cells. ITGA9 is highly expressed on neutrophils and is critical for neutrophil migration on vascular cell adhesion molecule-1 (VCAM-1).

Embodiments comprise administering to a subject in need thereof an ITGA9 inhibitor or a pharmaceutical composition comprising an ITGA9 inhibitor. “ITGA9 inhibitor” can refer to a molecule that directly or indirectly lowers or downregulates a biological activity of integrin alpha9 (ITGA9).

An ITGA9 inhibitor can be any member of a class of compounds (e.g., a small molecule, or an antibody or a fragment or derivative of such antibody such as a Fab fragment or a single chain antibody such as a scFv) that binds ITGA9 and inhibits a biological activity of a ITGA9 protein or a protein complex in which ITGA9 exerts its function. An ITGA9 inhibitor can also be any member of a class of compounds that decreases the expression of a nucleic acid encoding a ITGA9 protein (e.g., an inhibitory nucleic acid, RNAi, such as a small hairpin RNA). The skilled person is able to determine whether a compound can qualify as an ITGA9 inhibitor in such assay.

In embodiments, the ITGA9 inhibitor can comprise a small molecule compound. In embodiments, the inhibitor can be a small molecule inhibitor. The term “small molecule” can refer to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

For example, the ITGA9 inhibitor can comprise Macitentan. For example, Macitentan can comprise a structure according to the following, or a derivative or analog thereof:

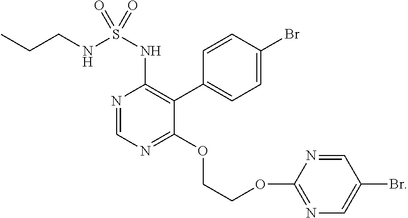 [SMILES: CCCNS(=O)(=O)Nc1ncnc(OCCOc2ncc(Br)cn2)c1-c1ccc(Br)cc1]

In embodiments, the ITGA9 inhibitor can comprise an antibody inhibitor, such as an anti-ITGA9 inhibiting antibody. The term “antibody” can refer to an immunoglobulin, whether natural or partly or wholly synthetically produced. An antibody can include any immunoglobulin, including antibodies and fragments thereof, that binds a specific epitope. The term encompasses polyclonal, monoclonal, recombinant, humanized, and chimeric antibodies. The term also covers any polypeptide or protein having a binding domain which is, or is homologous to, an antibody binding domain. CDR grafted antibodies are also intended by this term. As antibodies can be modified in a number of ways, the term “antibody” can be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567. For example, the inhibitor can comprise an anti-integrin alpha9 inhibiting antibody. For example, the anti-integrin alpha9 inhibiting antibody can comprise Y9A2 and 55A2C.

Fragments of a whole antibody can perform the function of binding antigens. Examples of binding fragments are (i) the Fab fragment consisting of VL, VH, CL and CHI domains; (ii) the Fd fragment consisting of the VH and CHI domains; (iii) the Fv fragment consisting of the VL and VII domains of a single antibody; (iv) the dAb fragment (Ward, E. S. et al, Nature 341, 544-546 (1989)) which consists of a VH domain; (v) isolated CDR regions; (vi) F(ab′) 2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) multivalent antibody fragments (scFv dimers, trimers and/or tetramers (Power and Hudson, J. Immunol. Methods 242:193-204 9 (2000)) (ix) bispecific single chain Fv dimers (PCT/US92/09965) and (x) “diabodies”, multivalent or multispecific fragments constructed by gene fusion (WO94/13804; P. Holliger et al Proc. Natl. Acad. Sci. USA 90 6444-6448, (1993)).

In embodiments, the inhibitor can be a nucleic acid molecule. For example, such nucleic acid inhibitors can include, but are not limited to, antisense oligonucleotides, siRNAs, shRNAs, microRNAs, aptamers, ribozymes and decoy oligonucleotides. A nucleic acid inhibitor can inhibit the expression of a target gene (e.g., ITGA9). Accordingly, in embodiments, a nucleic acid inhibitor comprises a sequence which is complementary to a portion of an mRNA encoding a target protein. In embodiments, a nucleic acid inhibitor comprises a sequence which is complementary to a portion of 5′ untranslated region of an mRNA encoding a target protein which also includes the start codon. In embodiments, a nucleic acid inhibitor comprises a sequence which is complementary to a portion of 3′ untranslated region of an mRNA encoding a target protein. In embodiments, complementarity is over a stretch of at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides.

A nucleic acid molecule can refer to DNA molecules and RNA molecules. A nucleic acid molecule can be single-stranded or double-stranded. In embodiments, the nucleic acid molecule is single stranded. In embodiments, the nucleic acid molecule is double-stranded DNA. As used herein, the term “isolated nucleic acid molecule” can refer to a nucleic acid molecule in which the nucleotide sequences are free of other nucleotide sequences, which other sequences can naturally flank the nucleic acid in human genomic DNA. Non-limiting examples of a nucleic acid molecule comprise a siRNA, miRNA, shRNA, antisense RNA, guide RNA (gRNA), single-guide RNA (sgRNA), modified forms thereof, or combination thereof.

In embodiments, the endothelin receptor inhibitor can be referred to as a “compound”. A compound can refer to any chemical entity, pharmaceutical, drug, and the like that can be used to treat or prevent a disease, illness, sickness, or disorder of bodily function (for example, a venous thromboembolic event). The term “compound” as used herein can include but is not limited to peptides, nucleic acids, carbohydrates, natural product extract libraries, organic molecules, such as small organic molecules, inorganic molecules, including but not limited to chemicals, metals, and organometallic molecules.

In embodiments, the compound can be an antagonist.

In embodiments, an inhibitor (e.g., an ITGA9 inhibitor) can act by a number of different pathways. For example, the inhibitor can bind to a ligand binding site on target protein and interfere with binding of the target protein to a ligand or receptor, bind to a nonligand binding site on target protein and interfere with binding of the ligand to a receptor, bind with a receptor and interfere with binding of the ligand to the receptor, or inhibit the expression of a polynucleotide (e.g., mRNA) expressing the target protein.

In embodiments, the inhibitor can inhibit binding with Vascular endothelial growth factor (VEGF), Vascular cell adhesion molecule 1 (VCAM-1), tenascin C, ostcopontin, fibronectin-EDA, thrombospondin-1 or disintegrin VLO5.

In embodiments, the inhibitor can inhibit the biological activity of its target protein by at least 5%, at least 10%, at least 15%, at least 20%, at least 30%, at least 40%, at least 50%, at least 60%, at least 70%, at least 75%, at least 80%, at least 85%, at least 90%, or at least 95% relative to a control. In embodiments, the inhibitor completely abrogates the biological activity of the target protein relative to a control. A control can comprise a sample that is not treated by an inhibitor.

The endothelin receptor inhibitors (e.g., the ITGA9 inhibitors) described herein can be formulated into pharmaceutical compositions using techniques and procedures well known in the art (see, e.g., Ansel Introduction to Pharmaceutical Dosage Forms, Fourth Edition 1985, 126). A “pharmaceutical composition” can refer to a composition or pharmaceutical composition for administration to a subject, such as a mammal, especially a human and that can refer to the combination of one or more agents described herein with a pharmaceutically acceptable carrier or excipient, making the composition suitable for diagnostic, therapeutic, or preventive use in vitro, in vivo, or ex vivo.

A “pharmaceutical composition” can be sterile, and can be free of contaminants that can elicit an undesirable response within the subject (for example, the agent(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intratracheal, intramuscular, subcutaneous, inhalational and the like.

Pharmaceutical compositions can further comprise an excipient, carrier, diluent, and/or adjuvant. A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that is useful in preparing a pharmaceutical composition that is safe, non-toxic and neither biologically nor otherwise undesirable, and can include an excipient, diluent, carrier, and adjuvant that is acceptable for veterinary use and/or human pharmaceutical use. See, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th cd., Lippincott, Williams, & Wilkins; and Handbook of Pharmaceutical Excipients (2000) A. H. Kibbe et al., eds., 3rd ed. Amer. Pharmaceutical Assoc. “A pharmaceutically acceptable excipient, diluent, carrier and/or adjuvant” as used in the specification and claims can include one and more such excipients, diluents, carriers, and/or adjuvants. For example, suitable excipient vehicles for the composition or pharmaceutical composition can be water, saline, dextrose, glycerol, ethanol, or the like, and combinations thereof.

In addition, the vehicle can contain minor amounts of pharmaceutically acceptable auxiliary substances, such as pH adjusting and buffering agents, tonicity adjusting agents, stabilizers, antioxidants, wetting agents and the like, are readily available to the public.

In embodiments, effective concentrations of the active agent (e.g., an endothelin receptor inhibitor) or pharmaceutically acceptable derivatives can be mixed with a suitable pharmaceutical carrier or vehicle. The inhibitors can be derivatized as the corresponding salts, esters, enol ethers or esters, acids, bases, solvates, hydrates or prodrugs prior to formulation, as described herein. The concentrations of the inhibitors in the compositions can be effective for delivery of an amount, upon administration, that treats, prevents, or ameliorates one or more of the symptoms of a disease, disorder or condition, such as venous thromboembolic events.

Compositions can include those that comprise a sustained release or controlled release matrix. In addition, embodiments can be used in conjunction with other treatments that use sustained-release formulations. As used herein, a sustained-release matrix is a matrix made of materials, such as polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho) esters, polypeptides, hyaluronic acid, collagen, chondroitin sulfate, carboxylic acids, fatty acids, phospholipids, polysaccharides, nucleic acids, polyamino acids, amino acids such as phenylalaninc, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix. In another embodiment, the pharmaceutical composition (as well as combination compositions) can be delivered in a controlled release system. For example, the composition or pharmaceutical composition can be administered using intravenous infusion, an implantable osmotic pump, a transdermal patch, liposomes, or other modes of administration. In one embodiment, a pump can be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions or pharmaceutical compositions can be part of a delayed-release formulation. Delayed-release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

As described herein, methods of preparing such pharmaceutical compositions are known, or will be apparent upon consideration of this disclosure, to those skilled in the art. Sec, e.g., Remington's Pharmaceutical Sciences, Mack Publishing Company, Easton, Pennsylvania, 17th edition, 1985. The composition or formulation to be administered will, in any event, contain a quantity of the composition or pharmaceutical composition adequate to achieve the intended state in the subject being treated.

The compositions can be a component of a pharmaceutical formulation. The pharmaceutical formulation can further contain known agents for the treatment of diseases such as venous thromboembolism, or symptoms thereof.

Embodiments also provides packaged composition(s) or pharmaceutical composition(s) for prevention, restoration, or use in treating the disease or condition. Other packaged compositions or pharmaceutical compositions can further include indicia including at least one of: instructions for using the composition to treat the disease or condition. The kit can further include appropriate buffers and reagents known in the art for administering various combinations of the components listed herein to the host.

Aspects of the invention are drawn to methods of preventing venous thromboembolic events in a subject. The terms “prevent,” “preventing” and/or “prevention” can refer to the prevention of the onset, recurrence or spread of a disease or disorder, such as a venous thromboembolic event or cancer-associated thrombosis, or of one or more symptoms thereof. In embodiments, the terms refer to the treatment with or administration of an agent (e.g., an endothelin receptor inhibitor) provided herein, with or without other additional active agents, prior to the onset of symptoms, such as to patients at risk of diseases or disorders provided herein. The terms encompasses the inhibition or reduction of a symptom of the disease. Non-limiting examples of thrombosis or thrombotic events include venous thrombosis, deep vein thrombosis, portal vein thrombosis, renal vein thrombosis, jugular vein thrombosis, Budd-Chiari syndrome, Paget-Schroetter disease, cerebral venous sinus thrombosis, pulmonary embolism, and arterial thrombosis.

Aspects of the invention are also drawn to methods of treating a venous thromboembolic event in a subject. The terms “treat,” “treatment,” and “treating” can refer to the management and care of a subject for the purpose of combating a condition, disease or disorder, such as a venous thromboembolic event or cancer-associated thrombosis, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active agents to prevent or reduce the risk of the onset of symptoms or complications.

Embodiments are also drawn to methods of alleviating a symptom of a venous thromboembolic event. The phrase “alleviating a symptom of” can refer to reducing or preventing a symptom associated with a venous thromboembolic event in a subject. Non-limiting examples of symptoms associated with a venous thromboembolic event can comprise leg pain or tenderness, leg swelling, skin that is warm to the touch, reddish discoloration or red streaks of the skin, unexplained shortness of breath, rapid breathing, chest pain, tachycardia, and light headedness.

In embodiments, endothelin receptor inhibitors (e.g., ITGA9 inhibitors) can be used to prevent or treat thrombosis or thrombotic events in a subject afflicted with a cancer. The terms “cancer” or “tumor” or “hyperproliferative disorder” can refer to the presence of cells possessing characteristics typical of cancer-causing cells, such as uncontrolled proliferation, immortality, metastatic potential, rapid growth and proliferation rate, and certain characteristic morphological features. Cancer can be associated with uncontrolled cell growth, invasion of such cells to adjacent tissues, and the spread of such cells to other organs of the body by vascular and lymphatic means. Cancer invasion occurs when cancer cells intrude on and cross the normal boundaries of adjacent tissue, which can be measured by assaying cancer cell migration, enzymatic destruction of basement membranes by cancer cells, and the like. In some embodiments, stage of cancer is relevant and such stages can include the time period before and/or after angiogenesis, cellular invasion, and/or metastasis. Cancer cells are often in the form of a solid tumor, but such cells can exist alone within an animal, or can be a non-tumorigenic cancer cell, such as a leukemia cell.

In embodiments, the cancer can be a solid tumor or a liquid cancer. A “solid tumor”, which can also be referred to as a “solid organ cancer”, can refer to an abnormal mass of tissue that does not contain cysts or liquid. A “non-solid tumor”, which can be referred to as a “liquid cancer,” can refer to neoplasia of the hemopoietix system, such as lymphoma, myeloma, and leukemia, or neoplasia without solid formation and with spread substantially.

Non-limiting examples of solid tumors comprise brain cancer, lung cancer (e.g., non-small cell lung cancer), liver cancer, hepatocellular carcinoma (HCC), esophageal cancer, cholangiocarcinoma, gallbladder carcinoma, stomach cancer, abdominal cancer, gastrointestinal cancer, gastric cancer, pancreatic cancer, renal cell carcinoma, renal cancer, bone cancer, breast cancer, ovarian cancer, uterine cancer, cervical cancer, endometrial cancer, colorectal cancer, colon cancer, rectal cancer, bladder cancer, superficial bladder cancer, prostate cancer, adrenal tumors, squamous cell carcinoma, neuroma, malignant neuroma, myoepithelial carcinoma, synovial sarcoma, rhabdomyosarcoma, gastrointestinal interstitial cell tumor, skin cancer, basal cell carcinoma, malignant melanoma, thyroid cancer, nasopharyngeal carcinoma, hemangioma, epidermoid carcinoma, head and neck cancer, glioma, or Kaposi's sarcoma. For example, the solid-tumor cancer comprises lung cancer (e.g., non-small cell lung cancer), prostate tumor, ovarian tumor, or pancreatic tumor.

Non-limiting examples of non-solid tumors or liquid cancers comprise leukemia, acute leukemia, chronic leukemia, chronic myelocytic leukemia, acute myelogenous leukemia, chronic myelogenous leukemia, chronic lymphocytic leukemia, acute lymphoblastic leukemia, T-cell leukemia, hairy cell leukemia, polycythemia, myelodysplastic syndrome, multiple myeloma, lymphadenoma, Hodgkin's lymphoma, and Non-Hodgkin's lymphoma.

Neoplasia can refer to the process of abnormal growth, for example, of a cell, tissue or organ. The growth is abnormal in that it is an uncontrolled, unrestrained and progressive multiplication of cells under conditions that cannot normally induce growth and/or that normally can prevent growth. Such abnormal growth can result in the generation of an abnormal mass, referred to as a neoplasm or tumor, which can be benign or malignant. Unlike hyperplasia, neoplastic proliferation persists even in the absence of the original stimulus.

A neoplastic disease can refer to any disease or disorder associated with neoplasia, whether benign or malignant. Examples of such diseases or disorders include, but are not limited to, malignant neoplastic diseases or disorders involving cancer, including tumor development, growth, metastasis and progression. For example, hematological malignancies affecting blood, bone marrow and/or lymph nodes, including leukemia, lymphoma and multiple myeloma, are types of neoplastic diseases or disorders.

The term “malignant”, as it applies to tumors, can refer to primary tumors that have the capacity to invade surrounding tissues and metastasize with loss of growth control and positional control. In contrast, benign tumors do not invade surrounding tissues or metastasize to other areas of an organism.

The terms “therapies” and/or “therapy” can refer to any protocol(s), method(s), compositions, formulations, and/or agent(s) that can be used in the prevention, treatment, management, or amelioration of a disease or disorder or a symptom associated therewith. In embodiments, the terms “therapies” and “therapy” can refer to biological therapy, supportive therapy, and/or other therapies useful in treatment, management, prevention, or amelioration of a disease or disorder or a symptom associated therewith known to one of skill in the art.

The terms “therapeutic agent”, and “therapeutic agents” can refer to any agent(s) which can be used in the prevention, treatment and/or management of a disease or disorder or a symptom associated therewith.

Embodiments as described herein can comprise administering to a subject a therapeutically effective amount of an endothelin receptor inhibitor, such as an ITGA9 inhibitor. For example, the ITGA9 inhibitor can comprise Macitentan. For example, Macitentan can comprise a structure according to the following, or a derivative or analog thereof:

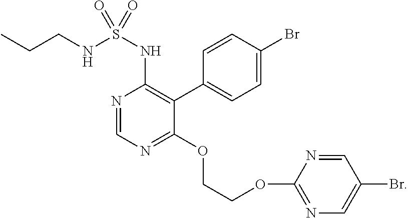 [SMILES: CCCNS(=O)(=O)Nc1ncnc(OCCOc2ncc(Br)cn2)c1-c1ccc(Br)cc1]

The term “therapeutic effect” can refer to a local or systemic effect in animals, particularly mammals, and more particularly humans, caused by a pharmacologically active substance. “Therapeutic effect” can refer to any substance intended for use in the diagnosis, cure, mitigation, treatment or prevention of disease or in the enhancement of desirable physical or mental development and conditions in an animal or human.

The term “therapeutically effective amount” can refer to that amount of an endothelin receptor inhibitor or pharmaceutical composition comprising the same being administered to a subject that will relieve to some extent one or more of the symptoms of the disease or condition being treated, and/or that amount that will prevent, to some extent, one or more of the symptoms of the condition or disease that the subject being treated has or is at risk of developing. For example, certain inhibitors encompassed by the methods described herein can be administered in a sufficient amount to produce a reasonable benefit/risk ratio applicable to such treatment. In an embodiment, therapeutically effective amount can refer to an amount needed to prevent or treat venous thrombosis, pulmonary embolism, or cancer-associated thrombosis.

In embodiments, a therapeutically effective amount of the endothelin receptor inhibitor (e.g., ITGA9 inhibitor) can comprise less than about 0.1 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, about 5.0 mg/kg, about 5.1 mg/kg, about 5.2 mg/kg, about 5.3 mg/kg, about 5.4 mg/kg, about 5.5 mg/kg, about 5.6 mg/kg, about 5.7 mg/kg, about 5.8 mg/kg, about 5.9 mg/kg, about 6.0 mg/kg, 6.1 mg/kg, about 6.2 mg/kg, about 6.3 mg/kg, about 6.4 mg/kg, about 6.5 mg/kg, about 6.6 mg/kg, about 6.7 mg/kg, about 6.8 mg/kg, about 6.9 mg/kg, about 7.0 mg/kg, about 7.1 mg/kg, about 7.2 mg/kg, about 7.3 mg/kg, about 7.4 mg/kg, about 7.5 mg/kg, about 7.6 mg/kg, about 7.7 mg/kg, about 7.8 mg/kg, about 7.9 mg/kg, about 8.0 mg/kg, 8.1 mg/kg, about 8.2 mg/kg, about 8.3 mg/kg, about 8.4 mg/kg, about 8.5 mg/kg, about 8.6 mg/kg, about 8.7 mg/kg, about 8.8 mg/kg, about 8.9 mg/kg, about 9.0 mg/kg, about 9.1 mg/kg, about 9.2 mg/kg, about 9.3 mg/kg, about 9.4 mg/kg, about 9.5 mg/kg, about 9.6 mg/kg, about 9.7 mg/kg, about 9.8 mg/kg, about 9.9 mg/kg, about 10.0 mg/kg, about 10.1 mg/kg, about 10.2 mg/kg, about 10.3 mg/kg, about 10.4 mg/kg, about 10.5 mg/kg, about 10.6 mg/kg, about 10.7 mg/kg, about 10.8 mg/kg, about 10.9 mg/kg, about 11 mg/kg, about 11.5 mg/kg, about 12.0 mg/kg, about 12.5 mg/kg, about 13.0 mg/kg, about 13.5 mg/kg, about 14.0 mg/kg, about 14.5 mg/kg, about 15.0 mg/kg, about 15.5 mg/kg, about 16.0 mg/kg, about 16.5 mg/kg, about 17.5 mg/kg, about 18.0 mg/kg, about 18.5 mg/kg, about 19.0 mg/kg, about 19.5 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 250 mg/kg, about 300 mg/kg, about 350 mg/kg, about 400 mg/kg, about 450 mg/kg, about 500 mg/kg, or more than 500 mg/kg of compound per body weight of a subject. For example, a therapeutically effective amount of the endothelin receptor inhibitor (e.g., ITGA9 inhibitor) can comprise between 2 mg/kg and 10 mg/kg.

The term “subject” or “patient” can refer to any organism to which aspects of the invention can be administered, e.g., for experimental, diagnostic, prophylactic, and/or therapeutic purposes. For example, subjects to which compounds of the disclosure can be administered include animals, such as mammals. Non-limiting examples of mammals include primates, such as humans. For veterinary applications, a wide variety of subjects will be suitable, e.g., livestock such as cattle, sheep, goats, cows, swine, and the like; poultry such as chickens, ducks, geese, turkeys, and the like; and domesticated animals for example pets such as dogs and cats. For diagnostic or research applications, a wide variety of mammals will be suitable subjects, including rodents (e.g., mice, rats, hamsters), rabbits, primates, and swine such as inbred pigs and the like.

Aspects of the invention can comprise administering to a subject pharmaceutical composition comprising an endothelin receptor inhibitor. The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. A “pharmaceutical composition” can be sterile and can be free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intratracheal, intramuscular, subcutaneous, by stent-eluting devices, catheters-eluting devices, intravascular balloons, inhalational and the like.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used herein can include one and more such excipients, diluents, carriers, and adjuvants.

Pharmaceutical composition can also be included, or packaged, with other non-toxic compounds, such as pharmaceutically acceptable carriers, excipients, diluents, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. For example, the pharmaceutically acceptable carriers, excipients, binders, and fillers for use in the practice of the invention are those which render the compounds of the invention amenable to intranasal delivery, oral delivery, parenteral delivery, intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers or silicone, and can be processed internally by the subject without affecting the effectiveness of the composition/formulation packaged and/or delivered therewith.

In embodiments, the pharmaceutical compositions can comprise pharmaceutically acceptable salts. Pharmaceutically acceptable salts can include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamineand other alkylamines, piperazine and tris(hydroxymethyl) aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Different forms of the pharmaceutical composition can be calibrated in order to adapt both to different subjects and to the different needs of a single subject. However, the pharmaceutical composition need not counter every cause in every subject. Rather, by countering the necessary causes, the pharmaceutical composition will restore the body to its normal function. Then the body will correct the remaining deficiencies.

For oral preparations, the pharmaceutical composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and optionally, with diluents, buffering agents, moistening agents, preservatives and flavoring agents.

Embodiments of the pharmaceutical composition can be formulated into preparations for injection by dissolving, suspending, or emulsifying them in an aqueous or non-aqueous solvent, such as vegetable or other similar oils, synthetic aliphatic acid glycerides, esters of higher aliphatic acids or propylene glycol; and optionally, with conventional additives such as solubilizers, isotonic agents, suspending agents, emulsifying agents, stabilizers and preservatives.

Embodiments of the composition or pharmaceutical composition can be utilized in aerosol formulation to be administered via inhalation. Embodiments of the composition or pharmaceutical composition can be formulated into pressurized acceptable propellants such as dichiorodifluoromethane, propane, nitrogen and the like.

Unit dosage forms for oral administration, such as syrups, elixirs, and suspensions, can be provided wherein each dosage unit, for example, teaspoonful, tablespoonful, tablet or suppository, contains a predetermined amount of the composition containing one or more compositions. Similarly, unit dosage forms for injection or intravenous administration can comprise the pharmaceutical composition as a solution in sterile water, normal saline or another pharmaceutically acceptable carrier.

As described herein, embodiments comprise administering to a subject in need thereof a therapeutically effective amount of an endothelin receptor inhibitor. The term “administer” or “administering” can refer to introducing a substance (e.g., an ITGA9 inhibitor) as described herein into a subject. One route of administration of the agent is intravenous administration.

The term “administering” can refer to introducing a substance into a subject. Any route of administration can be utilized including, for example, intranasal, topical, oral, parenteral, intravitreal, intraocular, ocular, subretinal, intrathecal, intravenous, subcutaneous, transcutaneous, intracutaneous, intracranial and the like administration. For example, “parenteral administration” can refer to administration via injection or infusion. Parenteral administration includes, but is not limited to, subcutaneous administration, intravenous administration, and intramuscular administration. For example, the inhibitor can be administered intranasally, by inhalation, intrapulmonarily, or by injection (e.g., intravenous or subcutaneous).

In embodiments, “administering” can also refer to providing a therapeutically effective amount of a formulation or pharmaceutical composition to a subject. The formulation or pharmaceutical compound can be administered alone, but can be administered with other compounds, excipients, fillers, binders, carriers or other vehicles selected based upon the chosen route of administration and standard pharmaceutical practice.

Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

In embodiments, the endothelin receptor inhibitor (e.g., ITGA9 inhibitor) can be administered alone, or can be administered as a pharmaceutical composition together with other compounds, excipients, carriers, diluents, fillers, binders, or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

Embodiments can be administered to a subject in one or more doses. The dose level can vary as a function of the specific composition or pharmaceutical composition administered, the severity of the symptoms and the susceptibility of the subject to side effects. Dosages for a given compound are readily determinable by a variety of means. For example, dosages can be determined by standard clinical techniques. In addition, in vitro or in vivo assays can be employed to help identify optimal dosage ranges. The precise dose to be employed can also depend on the route of administration and can be decided according to the judgment of the practitioner and each patient's circumstances.

In an embodiment, multiple doses of the pharmaceutical composition can be administered. The frequency of administration and the duration of administration of the pharmaceutical composition can vary depending on any of a variety of factors, e.g., patient response, severity of the symptoms, and the like. For example, in an embodiment, the pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. In an embodiment, the pharmaceutical composition can be administered 1 to 4 times a day over a period of time, such as 1 to 10-day time period, or longer than a 10-day period of time.

In embodiments, the pharmaceutical composition can be administered in combination with one or more additional active agents. For example, a first agent (e.g., a prophylactic or therapeutic agent) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second agent (e.g., a prophylactic or therapeutic agent) to a subject with a disease or disorder or a symptom thereof.

Unit dosage forms for oral administration, such as syrups, elixirs, and suspensions, can be provided wherein each dosage unit, for example, teaspoonful, tablespoonful, tablet or suppository, contains a predetermined amount of the composition containing one or more compositions. Similarly, unit dosage forms for injection or intravenous administration can comprise the composition or pharmaceutical composition in a composition as a solution in sterile water, normal saline or another pharmaceutically acceptable carrier.

Embodiments of the pharmaceutical composition can be formulated in an injectable composition in accordance with the disclosure. For example, injectable compositions are prepared as liquid solutions or suspensions; solid forms suitable for solution in, or suspension in, liquid vehicles prior to injection can also be prepared. The preparation can also be emulsified or the active ingredient (triamino-pyridine derivative and/or the labeled triamino-pyridine derivative) encapsulated in liposome vehicles in accordance with the present disclosure.

In an embodiment, the pharmaceutical composition can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Embodiments of the pharmaceutical composition can be administered to a subject in one or more doses. Those of skill will readily appreciate that dose levels can vary as a function of the specific composition or pharmaceutical composition administered, the severity of the symptoms and the susceptibility of the subject to side effects. Dosages for a given compound are readily determinable by those of skill in the art by a variety of means.

The duration of administration of the endothelin receptor inhibitor (e.g., ITGA9 inhibitor) or pharmaceutical composition, e.g., the period of time over which the agent or pharmaceutical composition is administered, can vary, depending on any of a variety of factors, including patient response. For example, the agent or pharmaceutical composition in combination or separately, can be administered over a period of time of about one day to one week, about one day to two weeks. The amount of the combination therapy and pharmaceutical compositions of the disclosure that can be effective in treating the condition or disease can be determined by standard clinical techniques. In addition, in vitro or in vivo assays can optionally be employed to help identify optimal dosage ranges. The precise dose to be employed can also depend on the route of administration, and can be decided according to the judgment of the practitioner and each patient's circumstances.

Embodiments as described herein provide methods for the administration of the active agent(s) (e.g., endothelin receptor inhibitor or ITGA9 inhibitor) to a subject using any available method and route suitable for drug delivery, including in vivo, in vitro and ex vivo methods, as well as systemic and localized routes of administration. Routes of administration include intranasal, intramuscular, intratracheal, subcutaneous, intra cerebroventricular, intradermal, topical application, intravenous, rectal, nasal, oral, and other enteral and parenteral routes of administration. Routes of administration can be combined or adjusted depending upon the agent and/or the intended effect. An active agent can be administered in a single dose or in multiple doses.

Embodiments of the composition or pharmaceutical composition can be administered to a subject using available conventional methods and routes suitable for delivery of conventional drugs, including systemic or localized routes. Routes of administration can include, but are not limited to, enteral administration, parenteral administration, or inhalation.

Other compositions, compounds, methods, features, and advantages of the disclosure will be or become apparent to one having ordinary skill in the art upon examination of the following drawings, detailed description, and examples. It is intended that all such additional compositions, compounds, methods, features, and advantages be included within this description, and be within the scope of the disclosure.

Compositions and pharmaceutical compositions as described herein can be administered locally or systemically. “Local administration” can refer to administering a composition or drug into a limited or partial anatomy space. Examples of local administration include but are not limited to intratumoral, intra-lymph node, intra-pleural space, intraperitoneal cavity and the like. “Systemic administration” can refer to administration of an anti-cancer agent such that the anti-cancer agent becomes widely distributed in the body in significant amounts and has a biological effect, e.g., its intended effect, in the blood and/or reaches its intended site of action via the vascular system. For example, systemic routes of administration include administration by (1) introducing the agent directly into the vascular system or (2) oral, pulmonary, or intramuscular administration wherein the agent is adsorbed, enters the vascular system, and is carried to one or more intended site(s) of action via the blood.

In embodiments, the subject has been administered a therapeutic regimen associated with increased incidence of a venous thromboembolic event. For example, a therapeutic regimen can comprise an anti-cancer therapeutic regimen (e.g., an immunotherapy, a chemotherapy, a radiotherapy, surgery).

In embodiments, the subject has been administered a thromboprophylaxis regimen. For example, a thromboprophylaxis regimen can comprise antithrombotic agents (e.g., warfarin, heparin, dabigatran, rivaroxaban, or apixaban, or any combination thereof).

Embodiments as described herein further comprises administering one or more additional active agents to a subject together with the endothelin receptor inhibitor (e.g., an ITGA9 inhibitor). Non-limiting examples of such additional active agents can comprise an antithrombotic agent (e.g., warfarin, heparin, dabigatran, rivaroxaban, or apixaban, or any combination thereof), a vaccine, an anti-inflammatory agent, anti-cancer agents, (e.g., an immunotherapy, a chemotherapy, a radiotherapy), a pain reliever, a steroid, or any combination thereof.

Aspects of the invention are also directed towards kits, such as kits comprising compositions as described herein for treating venous thromboembolic events. For example, the kit can comprise therapeutic combination compositions described herein.

In one embodiment, the kit includes (a) a container that contains the composition(s), such as that described herein, and optionally (b) informational material. The informational material can be descriptive, instructional, marketing or other material that relates to the methods described herein and/or the use of the agents for therapeutic benefit.

In an embodiment, the kit includes two or more agents. For example, the kit includes a first container that contains a composition that includes an endothelin receptor inhibitor (e.g., ITGA9 inhibitor), and a second container comprising a second active agent. In embodiments, the kit further comprises a third container comprising a third active agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the therapeutic combination composition, e.g., in a suitable dose, dosage form, or mode of administration (e.g., a dose, dosage form, or mode of administration described herein), to treat a subject who has a venous thromboembolism). The information can be provided in a variety of formats, include printed text, computer readable material, video recording, or audio recording, or information that provides a link or address to substantive material.

The composition in the kit can include other ingredients, such as a solvent or buffer, a stabilizer, or a preservative. The antagonist can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight. The kit optionally includes a device suitable for administration of the composition, e.g., a syringe or other suitable delivery device. The device can be provided pre-loaded with one or both of the agents or can be empty, but suitable for loading.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Methods of Preventing Venous Thromboembolism Employing Macitentan

The invention provides a method for inhibiting ITGA9 activity comprising contacting ITGA9 or a binding partner of integrin ITGA9 with an ITGA9 inhibitor, wherein the ITGA9 activity is inhibited. In aspects, the invention provides a new intervention by targeting integrin ITGA9 with a functional blocking inhibitor to prevent or treat venous thrombosis.

Venous thromboembolic (VTE) events including deep vein thrombosis (DVT) and pulmonary embolism (PE) occurs in up to 2 million Americans per year of which 10-30% of patients die within 30 days. Importantly, cancer patients have a 5- to 7-fold increased risk of developing VTE and those who develop VTE at diagnosis of cancer or within the year tend to have a significantly worse prognosis compared with cancer patients without VTE. While prophylactic anticoagulation reduces the rates of VTE in such high-risk patients, they only prevent approximately half of the VTE events, indicating the critical need for new and safe adjuvant treatments to reduce VTE burden.

In recent years, compelling evidence has emerged that implicates neutrophils in the initiation and pathogenesis of VTE. Integrin α9 (ITGA9) is highly expressed on neutrophils and is critical for neutrophil migration on vascular cell adhesion molecule-1 (VCAM-1). Using homology modeling, we determined that, Macitentan (endothelin receptor antagonist, an FDA approved drug used to manage the symptoms of pulmonary arterial hypertension) inhibits ITGA9-VCAM1 binding and reduced severity of VTE in mouse model of deep vein thrombosis (DVT).

As described herein, Macitentan (endothelin receptor antagonist, an FDA approved drug used to manage the symptoms of pulmonary arterial hypertension) can be useful for the prevention of VTE. Unlike other prophylactic anticoagulants, Macitentan is not associated with the risk of bleeding (safety) and can have additive/synergistic effect of existing drugs. For example, current guidelines recommend the use of low-dose of low-molecular weight (LMW) heparin or unfractionated heparin or direct oral anticoagulants (DOACs). Although the use of LMW and DOACs reduced the risk of VTE, they are associated with bleeding. Since Macitentan does not interfere directly with hemostasis mechanisms, it is not associated with bleeding.

Venous thrombosis is a major health issue that is associated with significant costs (˜$10 B/year) and increased morbidity and mortality. Venous thromboembolism or blood clots in the veins and when it migrates to the lungs, it causes pulmonary embolism (PE). Prophylactic anticoagulation is the current standard of care. However, use of prophylactic anticoagulation is associated with increased risk of bleeding and up to ˜30% of patients treated with anticoagulation can have recurrent VT, indicating need of new approaches. A need exists for new and effective therapeutic regimens for the prevention of VTE.

Coordinated interactions between neutrophil, platelet and endothelial cell contribute to the development of deep vein thrombosis (DVT). Neutrophils potentiate thrombus propagation through the formation of neutrophil-platelet aggregates, secreting inflammatory mediators, releasing tissue factor, generating free radicals, and producing neutrophil extracellular traps (NETs).

In aspects, the invention provides a method of inhibiting ITGA9 activity, comprising contacting integrin ITGA9 or a binding partner of integrin ITGA9 with Macitentan, wherein the ITGA9-VCAM1 binding is inhibited. Further, the invention provides a method for treating an integrin ITGA9-related condition in a mammal, comprising administering an effective amount of Macitentan to the mammal. Further still, the invention provides Macitentan for the prophylactic or therapeutic treatment of an integrin ITGA9-related condition. Further still, the invention provides a new intervention by targeting ITGA9 with a functional blocking inhibitor (e.g., peptides or small molecules) to prevent venous thrombosis.

Homology Modeling: In the absence of crystal structure of α9B1 we used homology modeling approach to build the atomistic model. The Crystal structure of α4β1 (PDB code: 3V4P) was used to model α9 subunit. The aligned model was superimposed on α4β1 and the coordinates of B1 along with divalent ions Ca2+ and Mg2+ were extracted and merged α9 subunit to make a full complex of α9B1. The final model was thoroughly optimized to remove the steric clashes.

Ligand preparation and Docking: FDA approved drugs (3113 in numbers) were taken from Selleckchem database. The docking studies were performed on PyRx software based on Autodock software.

Docking: The docking studies were performed on PyRx software based on Autodock software. The protein complex was converted to pdbqt format. The prepared ligands were docked in the complex with a grid considering the whole complex as a binding site in PyRx virtual screening tool using autodock Vina. The results were analyzed using Schrodinger academic version.

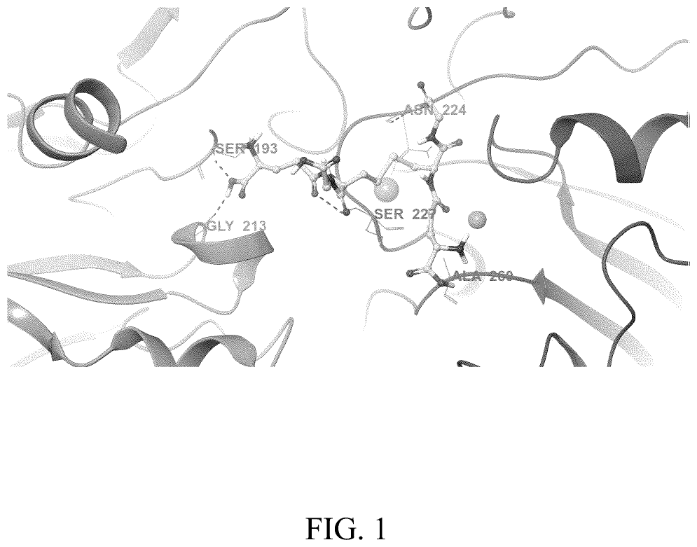

Ligand preparation and Docking: From the downloaded drug database we removed ions, small fragments (up to MW 250D) and large molecules (biologics). The final list was standardized to remove the salt followed by 3D preparation using OpenBabel version 2.3.2. The 3D compounds provided herein were imported to PyRx and minimized using UFF force field methods followed by conversion to pdbqt. The protein complex was converted to pdbqt format. The prepared ligands were docked in the complex with a grid considering the whole complex as a binding site in PyRx virtual screening tool using autodock Vina the constraints were applied to metal ions and residue SER132, TYR133, SER144 and ARG243. The results were analyzed using maestro academic version 12.0.012. Based on the docking score, number of interactions we filtered out 30 compounds. The pose of Macitentan with α9B1 is shown in FIG. 1.

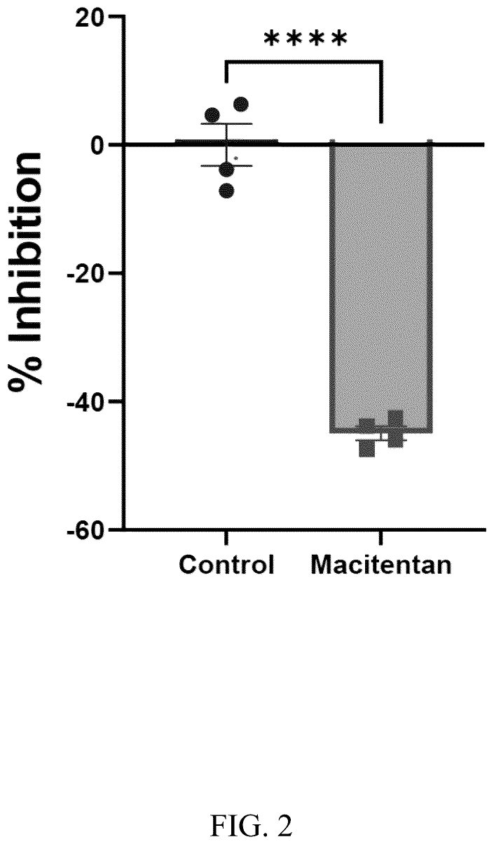

Turning next to FIG. 2, another aspect of the invention is shown as a ITGA9 binding assay. Briefly, purified human VCAM1 (R&D Systems) diluted to 5 μg/ml in TBS+ buffer (25 mM Tris pH 7.4, 137 mM NaCl, 2.7 mM KCl, 1 mM CaCl2, 1 Mm MgCl2, 1 mM MnC12) was added to wells of a 96-well transparent microtiter plate and incubated overnight at 4° C. Wells were washed 3 times with buffer and blocking buffer (TBS+ with 1% bovine serum albumin) was added followed by incubation for 1 hour at 37° C., and then washing. Recombinant human integrin ITGA9 (R&D Systems) was diluted to 1 μg/ml in TBS+/0.1% bovine serum albumin. Macitentan was diluted into the integrin solution and added to the washed ligand-coated plate according to a standard template with each sample repeated in triplicate. After incubation for 2 hours at room temperature, the plate was washed 3 times with 150 μl TBS+ buffer. To each well, biotinylated anti-β1 (catalog BAF1778, R&D Systems) at 1 μg/ml in TBS+/0.1% BSA was added, and the plate covered and incubated for 1 hour at RT. After washing the plate 3 times with TBS+ buffer, streptavidin conjugated horseradish peroxidase (R&D Systems) diluted in TBS+ blocking buffer was added to the wells and the plate incubated for 20 minutes at room temperature. The plate was washed 3 times with TBS+ buffer followed by addition of 50 μl TMB substrate (MilliporeSigma). After incubation for 20 minutes at RT, plates were read by colorimetric detection at 450 nm wavelength. Macitentan at 1 μM significantly inhibited ITGA9-VCAM1 binding. (FIG. 2).

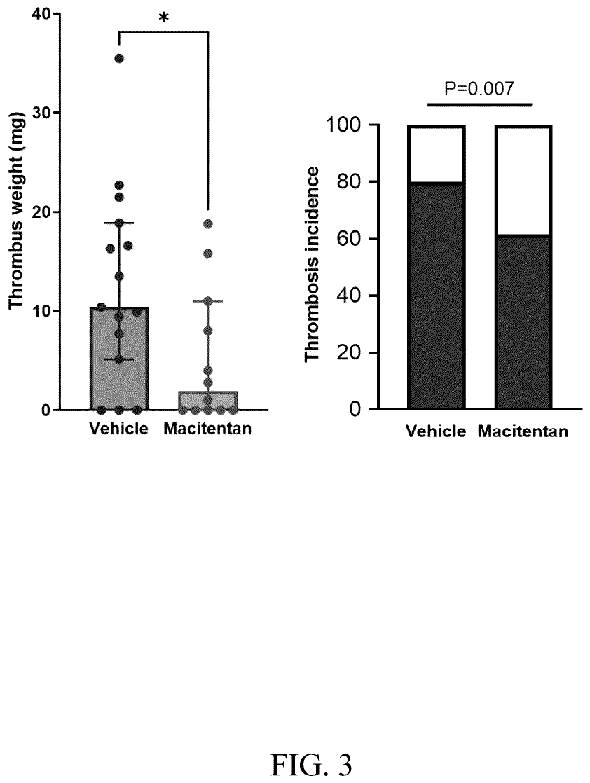

Next, we performed in vivo venous thrombosis using IVC stenosis (DVT surgery) in 30 wild-type mice and randomized them into control (vehicle, n=15) and treatment (Macitentan, 10 mg/kg, n=15) groups. The thrombus weight and incidence were measured at 24-hr. We observed significantly reduced thrombus weight and more importantly, thrombosis incidence was significantly reduced in Macitentan treated mice (FIG. 3).

### Example 2

### Research Strategy

### Significance

Importance of the problem: Venous thromboembolic (VTE) events, including deep vein thrombosis (DVT) and pulmonary embolism (PE), occur in up to 2 million Americans per year of which 10-30% of patients die within 30 days. The incidence of VTE increases sharply with age. Young individuals have very low VTE risk, but it increases to ˜1% per year in the elderly,¹ indicating that aging is one of the strongest and most prevalent risk factor for VTE. Similarly, patients with stroke are at risk for developing VTE events,²⁻⁶ which can result into a worse clinical outcome and is estimated to affect approximately 80,000 stroke patients each year in the United States. The overall prevalence of clinically evident DVT after acute stroke is approximately 10 percent.^{3,5,6} The prevalence of asymptomatic DVT is even higher.

Current strategies used to address the problem of interest and their limitations: Current guidelines recommend the use of low-dose of low-molecular weight (LMW) heparin or unfractionated heparin for the patients with acute ischemic stroke. Due to their higher baseline risk of VTE, older patients and patients with stroke can benefit more from antithrombotic prophylaxis. However, this potential benefit can be offset by a higher risk of bleeding complications associated with the use of antithrombotic prophylaxis in this population.⁷⁻¹¹ Notably, the use of anticoagulation is not recommended for initial 24 hours in patients who are treated with intravenous thrombolysis for acute ischemic stroke. While the prophylactic anticoagulation can reduce the rates of VTE in such high-risk patients, they only prevent approximately half of the VTE events,^{7, 8} indicating the critical need for new and safe adjuvant treatments to reduce VTE burden.

Scientific premise: The risk of DVT for patients who have had an acute ischemic stroke is close to that for patients undergoing major surgical procedures.²⁴ Without venous thromboembolism prophylaxis, up to 75% of patients with paralysis after stroke develop DVT and 20% develop PE,²⁵ which is fatal and causes up to 25% of early deaths after strokes.³ The onset of development of a DVT following stroke can be as early as on second day. Due to advances in the acute stroke care, many patients are now eligible for thrombolysis therapy (with rtPA) and mechanical thrombectomy. The inability to administer anticoagulation prophylaxis up to 24-hr due to the bleeding risk, combined with an early increase in VTE in the stroke patients, signifies the unique problem and highlights the critical need for new and safe interventions to reduce poststroke VTE burden.

### Rigor of Prior Research

Neutrophils in stroke and DVT: Stroke leads to increased output of proinflammatory myeloid cells including neutrophils as well as continuous basal hyperactivation of circulating neutrophils.^{26, 27, 28} Neutrophils are among the first cells in blood to respond after ischemic stroke. Coordinated interactions between neutrophils, platelets and endothelial cells contribute to the development DVT.^{15, 17, 19} The involvement of neutrophils in the pathogenesis of DVT is well-established. ¹²⁻¹⁹ Neutrophils potentiate thrombus propagation through the formation of neutrophil-platelet aggregates, secreting inflammatory mediators, releasing tissue factor, and producing neutrophil extracellular traps (NETs).^{14, 16} Given the basal hyperactivation of circulating neutrophils in stroke patients, a better understanding of molecular mechanisms that facilitate neutrophil adhesion can lead to the development of new and safe therapeutics to reduce the risk of VTE in the patients with stroke.

### Data

Increased risk of VTE in hospitalized patients with stroke: In a retrospective, case-control study, we utilized the data from the Nationwide Inpatient Sample (2016-2018). We evaluated the risk of VTE in hospitalizations due to ischemic stroke as compared to hospitalizations due to non-vascular neurological conditions (neurodegenerative disorders, cranial nerve disorders, headaches, movement disorders, multiple sclerosis).

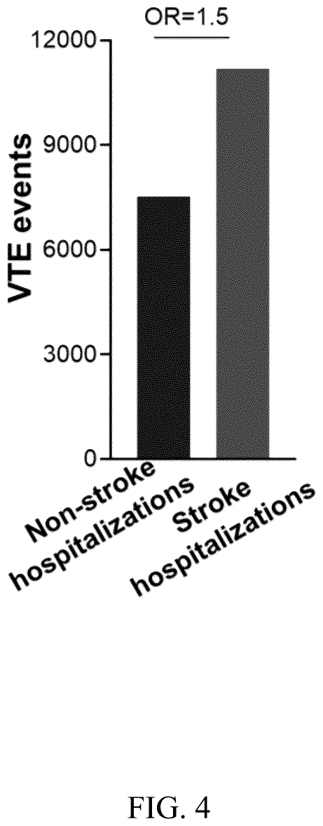

During the study period, there were 1,571,830 hospitalizations due to stroke and 1,483,625 hospitalizations due to non-vascular neurological conditions. After propensity score matching, 640,560 hospitalizations with stroke and corresponding well-matched controls were identified. At baseline, hospitalizations due to stroke were older and had a higher prevalence of comorbidities, as compared to the controls. The odds of VTE were significantly higher in stroke patients as compared to the propensity-matched controls (FIG. 4, odds ratio (OR) 1.50, 95% CI 1.40-1.60, P<0.001). In these data set, we observed that 80% of VTE events occurred within few days of stroke, indicating the presence of procoagulant state in stroke patients.

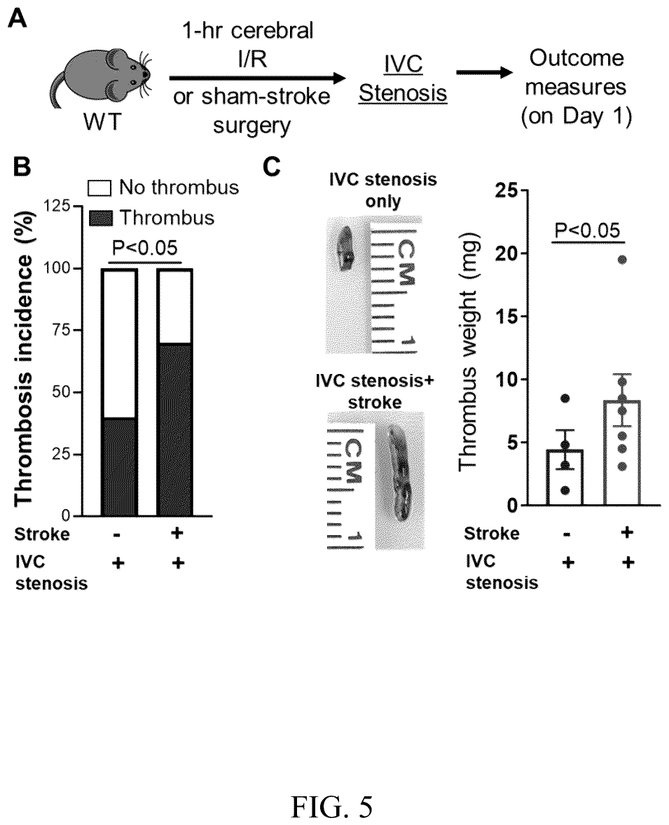

Increased DVT incidence in WT mice with stroke: We next evaluated susceptibility to DVT after cerebral ischemia/reperfusion injury in mice and compared it with mice with sham-stroke surgery. Partial ligation of inferior vena cava (IVC stenosis, without ligation of side branches) was performed in both the groups (following 1 hour of ischemia and 1 hour of reperfusion in the same mouse in stroke group and 2 hours after sham-stroke surgery in control group, FIG. 5, panel A). We evaluated thrombosis incidence on day 1 following stroke and found significantly increased DVT incidence in mice with stroke (7 out of 10) compared to mice without stroke (4 out of 10, P<0.05, FIG. 5, panel B). In line with these results, we also found a significantly increased in IVC thrombus weight in mice with stroke compared with mice without stroke (P<0.05, FIG. 5, panel C).

Human circulating neutrophils exhibit increased integrin α9 levels and are hyperactivated: Among the myeloid cells, neutrophils express high levels of integrin α9 compared with monocytes, and the expression level increases upon neutrophil activation.^{23, 29, 30} Platelets and lymphocytes do not express integrin α9.^{22, 23}

The significant increase in blood neutrophil counts following stroke occurs as a result of enhanced production, increased release from the bone marrow and spleen, and from a reduction in neutrophil apoptosis.^{26, 27} Within 15 minutes of ischemia, neutrophils express several adhesion molecules and by the 2 hours, neutrophil rolling and adhesion is present in the vasculature.^{31, 32} In a detailed and comprehensive analysis of peripheral blood neutrophils characteristics in a cohort of patients with acute ischemic stroke, Weisenburger-Lile et al²⁷ recently reported the basal hyperactivation of circulating neutrophils in the acute stroke phase (within 6 hours after stroke). The increased CD11b expression on neutrophils, increased circulating elastase levels in peripheral blood, and increased ROS production by unstimulated neutrophils were reported.

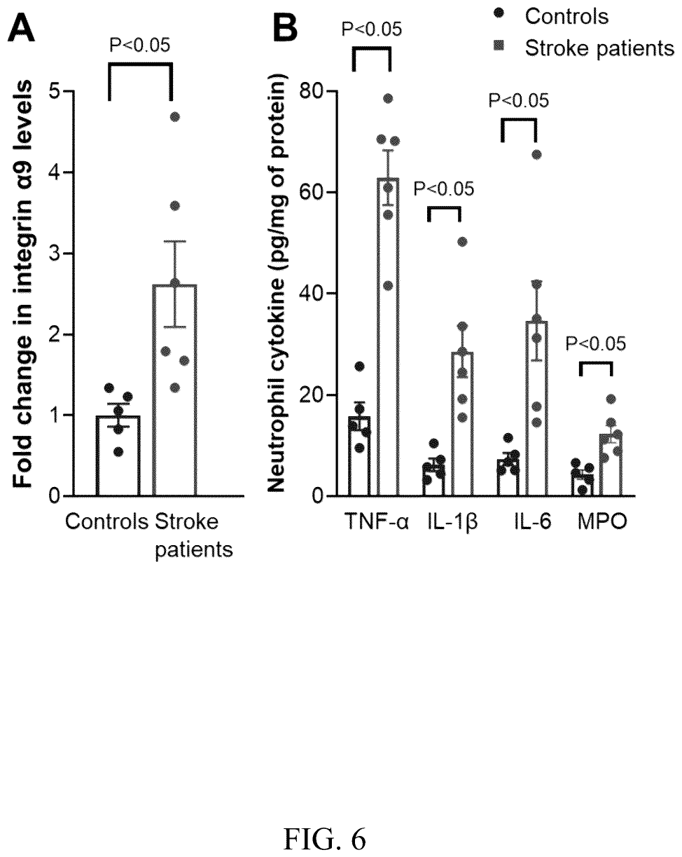

To determine whether integrin α9 expression increases on neutrophils after an acute ischemic stroke, neutrophils were isolated (within 6 hours of stroke) from the blood samples of patients with ischemic stroke. We observed significant increase in expression levels in ischemic stroke patients compared with healthy controls (FIG. 6, panel A). Next, we assessed inflammatory status of these neutrophils and found significantly increased levels of proinflammatory cytokines, and myeloperoxidase (MPO) levels (FIG. 6, panel B).

Next, we determined whether integrin α9 expression on peripheral neutrophils changes after acute ischemic stroke in mice. For this, neutrophils were isolated from the WT mice after 1 hour of ischemia followed by 3, 6, and 24 hours of poststroke. Littermate mice with sham surgery were used as controls. Integrin α9 expression was observed to be significantly upregulated following 3 and 6 hours of reperfusion. Together, these data indicate that ischemic stroke increases risk of DVT in humans and in mice and poststroke neutrophil exhibit increase integrin α9 levels.

Integrin α9-deficient neutrophils exhibit reduced adhesion following acute ischemic stroke: We next determined whether integrin α9-deficiency inhibits poststroke neutrophil adhesion to the endothelial cells. For this, peripheral neutrophils from myeloid-specific α9^{-/-} (α9^{fl/fl} LysMCre^{+/-}) and littermate control mice were isolated after 1 hour of ischemia and 3 hours of reperfusion and assayed for adhesion to TNF-α-activated mouse endothelial cells. We found that neutrophils from the myeloid-specific α9^{-/-} mice exhibited significantly reduced adhesion to the activated endothelium.³³

Integrin α9-deficient neutrophils exhibit reduced NETosis: In order to evaluate the role of integrin α9 in NETs formation; we quantified NETosis from the isolated neutrophils of α9^{fl/fl} and α9^{fl/fl} LysMCre^{+/-} mice, using thrombin-activated platelets as a stimulus. The percentage of cells releasing NETs was significantly reduced in α9^{fl/fl} LysMCre^{+/-} mice²², indicating that α9 can promote DVT by enhancing NETosis.

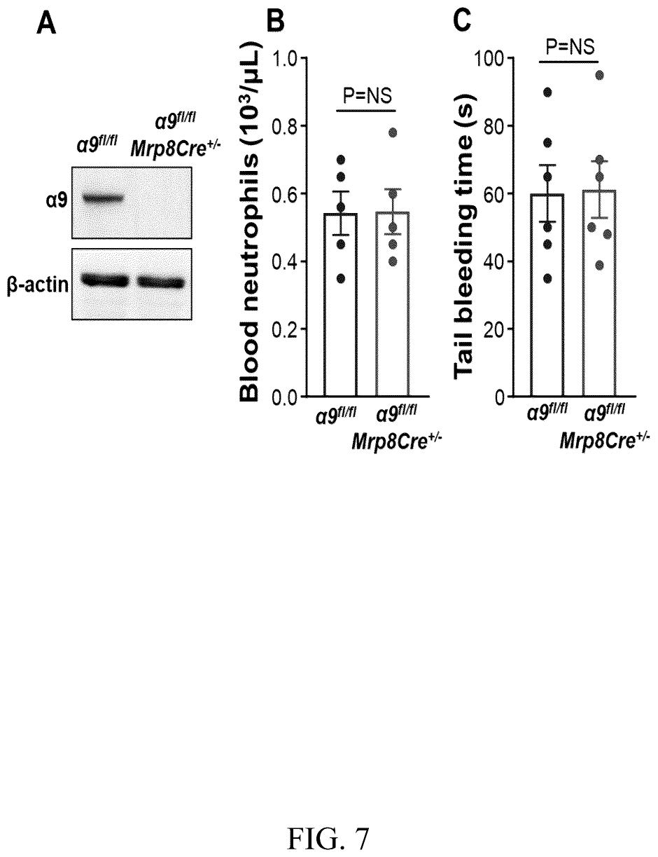

Generation and characterization of the neutrophil specific α9−/− mice: Integrin α9 is expressed on monocytes as well. In order to provide definitive role of neutrophil integrin α9 in pathogenesis of DVT, we generated new neutrophil specific α9−/− mice. For this, we crossed α9^{fl/fl} mice with Mrp8Cre⁺ mice (both on C57BL6/J background). FIG. 7, panel A shows integrin α9 expression on neutrophils isolated from bone marrow of α9^{fl/fl} and α9^{fl/fl} Mrp8Cre^{+/-} mice. Previously, it was shown that global deletion of α9 in mice results in granulopoiesis defect.³⁴ Here we found that mice with neutrophil specific deletion of integrin α9 did not have any defect in granulopoiesis. Blood neutrophil count (FIG. 7, panel B), total blood cell counts and tail bleeding time (FIG. 7, panel C) were comparable among the groups. Since β1 is the only known binding partner for α9, by knocking down α9 subunit from the neutrophils, it is expected that α9 mediated β1 signaling will be defective in neutrophils.

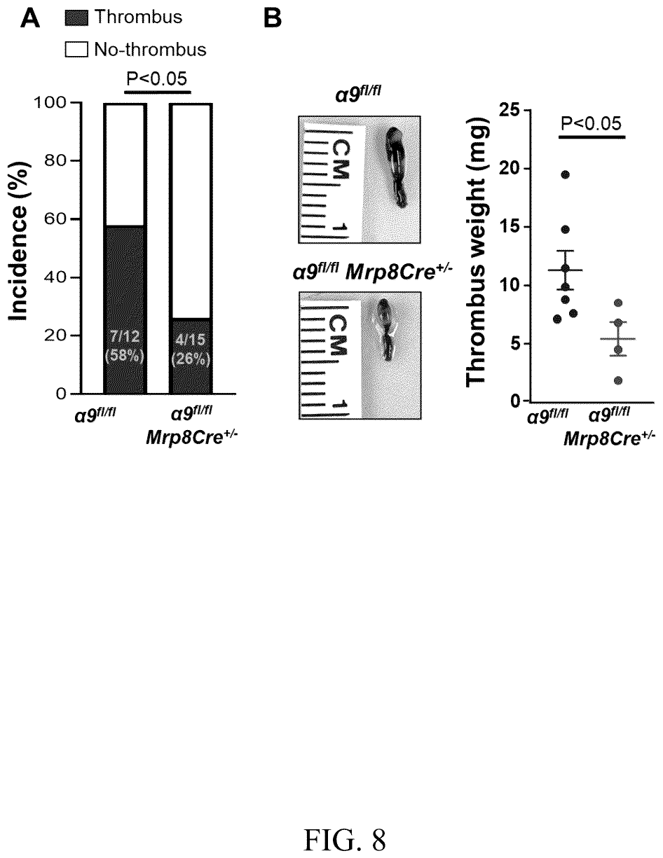

Neutrophil-specific α9^{-/-} mice were less susceptible to DVT, without affecting tail-bleeding time: We next evaluated DVT outcome in neutrophil-specific α9^{-/-} mice (in absence of stroke). For this, α9^{fl/fl} and α9^{fl/fl} Mrp8Cre^{+/-} mice (littermates) were subjected to IVC stenosis model (without side branch ligation). The experimenter was blinded for the genotype of the mice. Thrombi were harvested on day 2, after the surgery. We found that the DVT incidence was significantly reduced in α9^{fl/fl} Mrp8Cre^{+/-} mice (26% DVT incidence compare with 58% DVT incidence in controls, P<0.05, FIG. 8, panel A). Consequently, a marked decrease in thrombus weight was also observed in α9^{fl/fl} Mrp8Cre^{+/-} mice (P<0.05 versus α9^{fl/fl} mice, FIG. 8, panel B).

Our studies indicate:
1. Ischemic stroke significantly increases DVT risk.
2. Neutrophil integrin α9 expression increases within few hours of stroke in humans and in mice.
3. Integrin α9-deficient neutrophils exhibit reduced adhesion to the activated endothelium and exhibit reduced NETosis.
4. Neutrophil-specific α9−/− mice exhibit reduced DVT.

Without wishing to be bound by theory, our studies indicate that integrin α9 promotes poststroke neutrophil adhesion and NETosis, both of which are important mediators of venous thrombosis. We will use pharmacological inhibitors of the integrin α9 signaling pathway to further dissect molecular mechanisms involved in regulation of poststroke DVT. Specifically, we will evaluate whether integrin α9-FAK-SRC-Caspase-1 axis promotes NLRP3 inflammasome activation and NETosis. Using, chimeric mice (α9^{fl/fl} Mrp8Cre^{+/-} bone-marrow transplanted on Fn-EDA^{+/+} background), we will evaluate the underlying mechanisms that contribute to neutrophil adhesion and DVT.

Neutrophils represent the majority of white blood cells in humans (50-70%), but are less common in mice (10-30%). Accordingly, we will use blood sample from the controls and stroke patients to evaluate venous thrombosis.

The use of anticoagulation is not recommended in stroke patients treated with thrombolytics (rtPA) for initial 24 hours, during which neutrophil hyperactivation and increased adhesion can initiate venous thrombosis. We validate that inhibiting integrin α9 will reduce in vitro thrombosis using blood samples from the stroke patients treated with thrombolytics. For this we will utilize endothelial cells coated, custom-made microfluidic device that mimics stenosed veins. These experiments will confirm the relevance of targeting integrin α9 in reducing poststroke DVT.

Significance of the research contribution: Theoretically, morbidity and mortality from DVT following acute ischemic stroke can be reduced by more aggressive use of thromboprophylaxis. However, data from clinical trials indicate that the risk of major intracranial bleeding with early anticoagulation can outweigh the benefit, especially in the stroke patients treated with thrombolytic therapy.⁹ Without wishing to be bound by theory, the outcome of this proposal will provide new molecular mechanisms that can be targeted to reduce the DVT risk following ischemic stroke, without the risk of major bleeding. The proposal will also provide insights into new mechanisms by which neutrophil integrin α9 promotes DVT. Finally, the microfluidic studies using blood samples from the patients with stroke, will provide translational insight for targeting integrin α9 to reduce the risk of DVT in such patient population.

Advantages of targeting neutrophil integrin α9 to reduce poststroke VTE burden: Therapeutic targeting neutrophil adhesion molecules are associated with neutropenia and increased incidence of infections. For example, Danirixin (CXCR2 antagonist) was recently evaluated in double-blind, placebo-controlled phase IIb study for the treatment of patients with mild-to-moderate COPD. In this trial, there was an increased incidence of exacerbation in the danirixin-treated groups and an increased number of pneumonias in participants treated with danirixin 50 mg.²¹ Similarly, treatment with another CXCR2 antagonist, MK-7123 caused a dose-dependent decrease in absolute neutrophil count and increase in plasma C-reactive protein and fibrinogen levels.²⁰ Other adhesion molecules such as β2 integrins (CD11/CD18), PSGL-1 (CD162), L-selectin (CD62L) are expressed on most circulating leukocytes, and hence inhibition of these molecules can affect innate immunity.

In contrast, integrin α9 is highly and uniformly expressed on neutrophils while expression was not detected on lymphocytes and platelets.^{22, 23} Integrin α9 is expressed at low levels on monocytes.²³ In a recent Phase 2 trial, a humanized mAb against integrin-α9 was well-tolerated and safe overall, in patients with arthritis.³⁶ With single dose of this antibody, sustained plasma levels were observed up to 4-week without affecting neutrophil counts.³⁶ In our mouse study, we did not observe reduction in neutrophils counts upon treatment with anti-integrin α9 antibody. Interestingly, inhibition of integrin α9 has been shown to advantageous in stroke, cancer, autoimmune disease, and inflammation. Because of the acute nature of the treatment, the possibility of unexpected and adverse physiological side effects of targeting integrin α9 with a specific inhibitor is minimal.

The proposal is clinically significant and conceptually innovative due to the following reasons:

(1) The research uses a conceptual innovative approach that leverages a specific and low-risk pathway to develop a new therapeutic strategy to reduced VTE burden in patients with ischemic stroke. Without wishing to be bound by theory, a single dose of integrin α9 inhibitor can be incorporated with current thromboprophylaxis regimens in patients with ischemic stroke. Since the use of anticoagulation is not recommended for initial 24 hours in patients who are treated with intravenous thrombolytics, without wishing to be bound by theory, integrin α9 inhibition will reduce DVT severity in such case. We will validate that targeting integrin α9 will reduce susceptibility to DVT following acute ischemic stroke with minimal bleeding risk.

(2) Another important aspect of this proposal is the innovative approach to evaluate efficacy of integrin α9 inhibition on poststroke DVT severity using clinically relevant models, use of both male and female mice and also in presence of preexisting comorbidities. This will confirm optimal scientific rigor will ensure reproducibility.

(3) State-of-the art techniques (intravital microscopy, magnetic resonance, and laser speckle imaging) and new mutant strain (neutrophil-specific α9−/− mice) will be utilized to determine the physiological relevance of integrin α9 in post-stroke DVT progression.

(4) Further, we will test the in vitro efficacy of anti-integrin α9 antibody in microfluidic flow-chamber assay using blood samples from the controls and stroke patients.

### Approach

There are no previous reports which specifically evaluated the cellular and molecular mechanisms that promote the poststroke DVT. In our study (FIG. 8), utilizing healthy young male mice without stroke, we observed that neutrophil-specific α9−/− mice were less susceptible to DVT.

### Strategies to Ensure Rigor of the Research:

We will adopt a rigorous preclinical study design by using randomization, blinding, prior sample size determination and using predefined inclusion/exclusion criteria. We have applied rigorous methodology to our prior studies^{37, 38}, using a CONSORT-like clinical trial approach which will have transparency in reporting with minimum biases. Aging is the strongest and most prevalent risk factor for VTE. To mimic that clinical condition, we will utilize 18-22 months old mice which correspond to 56-69 years of humans.

Sex and other biological variables: We recently reported sex- and age-specific differences in mice following acute ischemic stroke.³³ Consequently, we will use 8-12-week (young) male and female mice and 18-22-month (aged) male and female mice. We will breed new neutrophil-specific α9^{-/-} (α9^{fl/fl} Mrp8Cre^{+/-}) mice and their littermate control (α9^{fl/fl} Mrp8Cre^{-/-}) to control for microbiota, diet, temperature, and humidity, thus making sure to minimize any biological variables affecting the results.

### Aim 1: Determine the Mechanistic Role of Neutrophil Integrin α9 in Promoting DVT in the Context of Stroke and Aging.

Rationale: Stroke leads to increased output of proinflammatory myeloid cells including neutrophils as well as continuous basal hyperactivation of circulating neutrophils.^{26, 27, 28} Coordinated interactions between neutrophils, platelets and endothelial cells contribute to the development DVT.^{15, 17, 19} Our data indicates that neutrophil integrin α9 expression increases within few hours of stroke in humans and in mice and integrin α9-deficient neutrophils exhibit reduced adhesion to the activated endothelium and exhibit reduced NETosis. While the potential detrimental effects of neutrophils in the pathogenesis of DVT are well documented,^{15, 17, 19} the role of integrin α9 in the modulation of DVT in the context of stroke and aging remains unclear.

The main objective of this aim is to determine the mechanistic role of neutrophil integrin α9 in modulation of DVT in context of stroke and aging and to evaluate relevant molecular mechanisms. To attain this objective, we will validate that neutrophil-specific α9^{-/-} (α9^{fl/fl} Mrp8Cre^{+/-}) mice are less susceptible to DVT following acute ischemic stroke and in young and aged mice. In order to validate this, we will include littermate controls and neutrophil specific α9−/− mice of both sexes, young and aged, with and without stroke and treated with or without thrombolytic therapy.

Aim 1A: Validate that Neutrophil Integrin α9 Promotes DVT Following Stroke.

Experimental design: To validate that neutrophil-specific integrin α9^{-/-} (α9^{fl/fl} Mrp8Cre^{+/-}) mice are less susceptible to DVT following stroke, we will use both the stroke models, filament and embolic model. Mice will be subjected to 1-hr of cerebral ischemia. Reperfusion will be achieved by filament removal of by the infusion of rtPA, and while the other group will be without reperfusion. To evaluate DVT, IVC stenosis will be performed. Details of surgical models are provided in vertebrate animal section. PI has prior experience with both the models of stroke (filament and embolic) and models of DVT as evident by prior publications.^{33, 39-41}

Outcome measures: 1) IVC thrombus size and thrombosis incidence, analyzed by MRI at day-1 and 2. Neutrophil adhesion will be evaluated 4-hr after stenosis using intravital microscopy. 2) Venous blood flow at the stenosis site will be evaluated using laser-speckle imaging. 3) Neutrophil adhesion at the stenosis site using intravital microscopy 4) IVC clot content and clot properties will be evaluated using immunohistochemistry and scanning electron microscopy.

DVT after sham or stroke-surgery will be evaluated in littermate controls (α9^{fl/fl} Mrp8Cre+, groups 1 to 3) and in experimental mice (α9 ft/fMrp8Cret′, groups 4 to 6). Following Table 1 summarizes the different group comparisons and possible interpretations:

TABLE 1

| Group Comparison | Question that will be answered: |
|---|---|
| 1 versus 2 | What is the effect of cerebral ischemia/ |
| | reperfusion injury on DVT outcome? |
| 1 versus 3 | What is the effect of cerebral ischemia |
| | (without reperfusion) on DVT outcome? |
| 1 versus 4 | What is the effect of neutrophil |
| | integrin α9 deletion on DVT |
| | outcome? (in absence of stroke) |
| 2 versus 5 | What is the effect of neutrophil |
| | integrin α9 deletion on DVT |
| | outcome following cerebral I/R injury? |
| 3 versus 6 | What is the effect of neutrophil |
| | integrin α9 deletion on DVT |
| | outcome following cerebral ischemia? |

Aim 1B: Validate that Neutrophil Integrin α9 Promotes DVT in Aged Mice

The incidence of VTE increases sharply with age. While it is very rare in young individuals (<1 per 10 000 per year) but increases to ˜1% per year in the elderly, indicating that aging is one of the strongest and most prevalent risk factors for VTE. Aging is also significant risk factor for the acute ischemic stroke. To evaluate the role of integrin α9 in modulation of DVT following stroke in the context of aging, we will include aged (18-22 months) mice in our study. Aged littermate controls and neutrophil-specific integrin α9^{-/-} (α9^{fl/fl} Mrp8Cre^{+/-}) mice with and without stroke will be subjected to IVC stenosis models and DVT will be evaluated following stroke as described in Aim 1a. This research strategy (Aim 1a and 1b) will define the role of neutrophil integrin α9 in promoting DVT in the context of stroke and aging.

Cre-only control: α9^{fl/fl} Mrp8Cre^{+/-} transgenic mice expresses Cre recombinase under the control of MRP8 promoter that directs bicistronic Cre protein expression to granulocytes progenitors. MRP8-Cre mice show ˜80% deletion in neutrophils, with little deletion in other white blood cells.⁴² Although the Cre-lox system provides remarkable control over gene expression and the deletion is limited to the target cells (neutrophils of case of Mrp8Cre), some reports indicate ‘ectopic’ expression of Cre recombinase. In order to control for non-specific effects of Mrp8Cre recombinase, we will include Cre only control (Mrp8Cre^{+/-}) mice. DVT susceptibility after IVC stenosis will be evaluated in α9^{fl/fl} Mrp8Cre^{+/-} and Mrp8Cre^{+/-} mice following stroke.

Tail-bleeding experiments: Since platelets do not express integrin α922, the possibility of bleeding phenotype is minimal upon integrin α9 inhibition. To evaluate bleeding tendency, in complementary experiments, we will evaluate the tail bleeding time in controls and neutrophil specific α9^{-/-} mice following stroke.

End-points evaluation: We have optimized following techniques to evaluate venous thrombosis:

1. MRI, scans will be performed on days 1 and 2 after IVC stenosis. Anesthetized mice will be placed in 7.0 Tesla MRI (Agilent Technologies Inc., Santa Clara, CA, USA) bore with two channel receive-only surface coil. The imaging parameters of the spoiled gradient echo sequence will be performed as per published protocols.⁴³

2. Laser-speckle imaging, moorFLPI-2 imager from Moor instruments will be used to determine early changes in IVC blood flow. Speckle imaging in anesthetized mice will be obtained using temporal filter (250 frames, 10 sec/frame) at 0.1 Hz at baseline and 1, 2 and 6-hr post IVC stenosis.

3. Intravital microscopy will be utilized to visualize neutrophil adhesion at the stenosis site, 4-hr post stenosis. Nikon upright microscope with CF1 Fluor 10× and 20× water immersion objective was used to visualize FITC-Ly6G labelled neutrophils. The movies were recorded through a high-speed EM camera and evaluated off-line using a Nikon computer-assisted image analysis program.

4. IVC clot content and clot properties: Neutrophil content (Ly6G; 1A8 clone), platelet content (CD41), fibrin content (fibrin antibody 59D8) and NETs accumulation (with anti-citrullinated histone H3) in the thrombus will be evaluated by immunohistochemistry and Western blot. Clot properties will be evaluated by measuring total RBC content and fibrin fiber thickness using scanning electron microscopy. Inflammatory cytokines (CXCL1, CXCL5, IL-1β, TNF-α, IL6) levels in the plasma and in thrombus lysates will be quantified using ELISA.

Aim 1C: Evaluate Molecular Mechanisms by which Integrin α9 Promotes DVT

Integrin α9 activation is associated with modulation of signaling from several kinases including Src family kinases and focal adhesion kinase (FAK) in cancer cells.⁴⁴ Importantly, FAK and SRC kinases are involved in neutrophil adhesion and NETosis.⁴⁵⁻⁴⁸ Recently it has been reported that NETosis and caspase-1 activation are tightly interrelated processes, which cooperatively promote DVT.⁴⁹ The main objective of this aim is to elucidate the mechanisms by which neutrophil integrin α9 promotes DVT following stroke. Without wishing to be bound by theory, integrin α9 modulates DVT by promoting FAK-SRC dependent caspase-1 activation and subsequent inflammasome activation and NETosis.

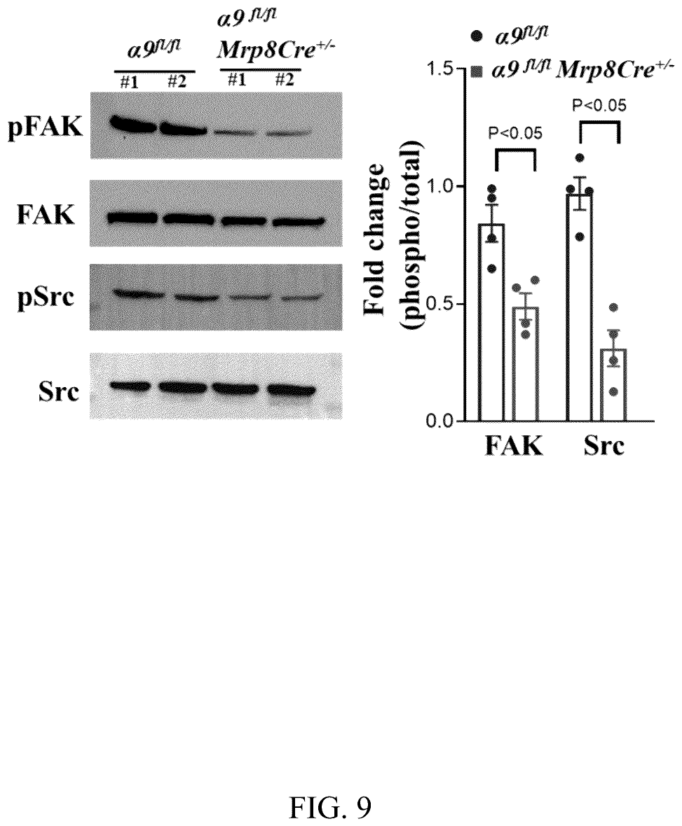

Experiment 1: Evaluation of integrin α9 downstream signaling events following neutrophil activation: In our study, we found significant reduction in pFAK and pSrc, upon stimulation with PMA (20 ng/ml), in neutrophils isolated from α9^{fl/fl} Mrp8Cre^{+/-} mice as compared with neutrophils from control α9^{fl/fl} mice (FIG. 9).

To evaluate downstream signaling pathway, we will use the primary neutrophils from control α9^{fl/fl} mice and α9^{fl/fl} Mrp8Cre^{+/-} mice. Neutrophils (2×106 cells/mL) will be added to 12-well plates (precoated with 5 μg/ml cellular fibronectin) and stimulated with PMA (20 ng/ml) and 3 mM ATP. Cells were incubated for the required time ranging from 10 to 60 minutes, washed with PBS to evaluate 1) phosphorylation status of FAK and Src using western blotting in cell lysate, 2) Caspase-1 activation using fluorometric caspase-1 assay kit (Abcam cat #39412) in cell lysates, 3) Inflammatory cytokines by ELISA 4) NLRP3 inflammasome status using western blot and immunofluorescence. Inflammasome images will be obtained as 0.5-1 μm z-stacks and presented as maximum intensity projections following stating with anti-caspase-1 antibody (Abcam cat #ab138483), Anti-NLRP3 antibody (Abcam cat #ab4207) and anti-Asc antibody (Adipogen cat #AL177), and 5) NETosis will be evaluated using anti-Histone H3 (citrulline R2+R8+R17) antibody (Abcam cat #ab5103).

Experiment 2: Evaluate the role of FAK/Src pathway in integrin α9 mediated downstream signaling: For this, we will use the integrin α91-specific ligand, TNfn3RAA. It is a recombinant form of the third fibronectin type III repeat of chicken tenascin-C containing alanine (A) substituted for glycine (G) and aspartate (D) residues of the common integrin-binding domain RGD. Isolated human and wild-type mouse primary neutrophils (2×106 cells/mL) will be added to 12-well plates (precoated with TnfnRAA and cellular fibronectin, 5 μg/ml) and stimulated with PMA (20 ng/mL) and 3 mM ATP. Cells were incubated for the required time ranging from 10 to 60 minutes. To evaluate the role of FAK/Src pathway, pharmacological inhibitors will be used. For FAK inhibition, cells will be pretreated (30 min before stimulation) with PF-562271 (10 nM, Sigma cat #PZ0387). For Src inhibition, cells will be pretreated (30 min before stimulation) with InSolution PP1 Analog (200 nM, Sigma cat #529605-M). These inhibitors will be used separately and in combination to evaluate specific effect of FAK and Src on neutrophil activation. Endpoints to evaluate neutrophil inflammasome activation and NETosis, will be similar to the experiment #1.

PF-562271 displayed robust inhibition in cell-based assay measuring phospho-FAK with an IC50 of 5 nM and it inhibits FAK phosphorylation in vivo in a dose-dependent fashion which was sustained for >4 hours with a single dose of 33 mg/kg.50 Similarly, PP1 is a potent, and selective inhibitor of the Src family of protein tyrosine kinases with IC50 of 170 nM and in vivo dose of 1.5 mg/kg produces significant inhibition of Src.⁵¹

Experiment 3: Evaluate in vivo significance of integrin α9-FAK/Src_pathway poststroke: For this, control α9^{fl/fl} mice and α9^{fl/fl} Mrp8Cre^{+/-} mice will be subjected to stroke as mentioned in Aim 1a. Peripheral neutrophils will be isolated after 6, 12 and 24 hours post stroke. Isolated neutrophils (2×106 cells/mL) will be treated with FAK inhibitor (PF-562271.10 nM,) and Src inhibitor (PP1, 200 nM) for 1 hour followed by evaluation of neutrophil inflammasome activation and NETosis, as mentioned in experiment #1.

Next, in a separate set of control α9^{fl/fl} mice and α9^{fl/fl} Mrp8Cre^{+/-} mice with stroke, FAK and Src inhibitors will be injected intraperitoneally (33 mg/kg and 1.5 mg/kg, respectively, at the time of reperfusion). DVT after IVC stenosis will be evaluated as mentioned in Aim 1a.

Experiment 4: Elucidate the mechanistic role of integrin α9/Fn-EDA axis in modulation of DVT following stroke: Fn-EDA is a prothrombotic and pro inflammatory ligand of integrin α9. In the flow chamber assay, the presence of Fn-EDA in the whole blood produced larger thrombi, exhibited increased mortality in collagen-epinephrine induced pulmonary thromboembolism, and accelerated arterial thrombus formation in the FeCl3 injury-induced mesenteric artery thrombosis models.^{52, 53} First, to investigate the role of Fn-EDA in DVT, we subjected Fn-EDA^{+/+} and control mice to inferior vena cava (IVC) ligation (stasis model of DVT). Fn-EDA^{+/+} mice constitutively express Fn-EDA and exhibit increased plasma Fn-EDA levels. We found significantly increased thrombus weight and length in Fn-EDA^{+/+} mice, 2 days after IVC stenosis.

TABLE 2
Chimeric mice that will be subjected
to IVC stenosis following stroke.

| | Bone marrow donor | Neutrophil | |
|---|---|---|---|
| Recipient Mice | mice | Integrin α9 | Fn-EDA |
| Fn-EDA^{+/+} | α9^{flfl} | + | + |
| Fn-EDA^{+/+} | α9^{fl/f}lMrp8Cre^{+/-} | − | + |
| Fn-EDA^{-/-} | α9^{fl/fl} | + | − |
| Fn-EDA^{-/-} | α9^{fl/fl}Mrp8Cre^{+/-} | − | − |

To evaluate the in vivo mechanistic role of integrin α9/Fn-EDA axis in modulating DVT in the context of stroke, we will utilize chimeric mice (Table 2). Lethally irritated Fn-EDA^{+/+} and Fn-EDA/mice will receive bone-marrow (BM) cells from α9^{fl/fl} or α9^{fl/fl} Mrp8Cre⁺ mice. DVT endpoints will be compared between control Fn-EDA^{+/+} with α9^{fl/fl} BM (α9^{fl/fl} BM->Fn-EDA^{+/+}) versus Fn-EDA^{+/+} with neutrophil specific α9^{-/-} BM (α9fl/flMrp8Cre⁺ BM->Fn-EDA^{+/+}) mice. Specificity of the α9/Fn-EDA interaction in the modulation of DVT phenotype will be evaluated by comparing α9^{fl/fl} BM->Fn-EDA^{-/-} mice versus α9^{fl/fl} Mrp8Cre⁺ BM->Fn-EDA^{-/-} mice. DVT after IVC stenosis in mice with stroke using filament model will be evaluated in these chimeric mice. The magnitude of DVT (blood flow, clot size and thrombosis incidence) will be evaluated using state-of-the-art laser-speckle and MRI.

Statistical analysis, sample Size & power considerations: Shapiro-Wilk test will be used to check normality, and Bartlett's test will be used to check equal variance. The results will be considered significant at P<0.05. Normally distributed data will be analyzed by Student's t-test or two-way ANOVA followed by Sidak's multiple comparisons test and nonnormally distributed data will be analyzed using the Mann Whitney test (for two-group) or non-parametric two-way ANOVA followed by Fischer's LSD test. We performed a preliminary study of evaluating DVT susceptibility in control and experimental mice in absence of stroke. In this study, IVC thrombus weight in neutrophil-specific α9^{-/-} mice was 2.5 (SD 5.1) compared with 6.5 mg (SD 6.7) in control mice. As such, the mean difference between groups was 4 mg. There are 6 different mice groups. In order to control overall type I error rate under 5%, the significance level for each experiment is determined by the Bonferroni method at 0.05/6-0.008. Within each experiment, the power to detect a difference of 4 mg between group is calculated for the Dunnett's test. At overall significance level 0.05, the power to detect such a difference can be 80% with 25 mice in each group.

Results and interpretation: Without wishing to be bound by theory, neutrophil specific α9−/− mice will display significantly reduced DVT susceptibility following stroke in filament and embolic models. This effect will be preserved in aged male and female mice. Such results indicate integrin α9 contributes to DVT exacerbation and can be targeted to reduce DVT incidence in patients with acute ischemic stroke. In mechanistic studies, without wishing to be bound by theory, inhibition of integrin α9 downstream signaling (FAK/Src) pathway will reduce neutrophil inflammasome activation and NETosis in α9^{fl/fl} mice but will not have additional effects in α9^{fl/fl} Mrp8Cre^{+/-} mice. Adoptive transfer experiments will reveal the contribution of Fn-EDA in integrin α9-mediated post-stroke DVT. Without wishing to be bound by theory, poststroke DVT will be attenuated in α9^{fl/fl} Mrp8Cre+BM->Fn-EDA^{+/+} mice but will not have an additive effect in α9^{fl/fl} Mrp8Cre BM->Fn-EDA^{-/-} mice.

The experimental models are widely accepted in the field54-56 and these protocols have been optimized as evident by prior publications.^{33, 37, 39-41, 57}

### Aim 2: Determine the Translational Impact of Inhibiting Integrin α9 on Venous Thrombosis:

We have recently reported that targeting integrin α9 with a specific antibody 55A2C significantly improved functional outcomes in preclinical stroke models.³³ The objective of this aim to is determine therapeutic potential and translational feasibility of blocking integrin α9 to reduce DVT severity in the context of stroke and aging. Without wishing to be bound by theory, blocking integrin α9 will reduce neutrophil adhesion and subsequent DVT. The anti-α9 antibody (55A2C) is known to inhibit the binding of α9/NIH cells to the synthetic peptides AEIDGIEL, the sequence similar to the EDGIHEL sequence present the Fn-EDA segment.⁵⁹

Aim 2.1 Determine therapeutic potential of anti-integrin α9 antibody in post-stroke DVT: For this, we will utilize a well-characterized anti-integrin α9 antibody (55A2C, at dose of 2, 5 and 10 μg/g, I.V.). We currently have material transfer agreement with the company that provides this antibody. Experimental design for aim 2.1 antibody has been reported to attenuate arthritis and multiple sclerosis.⁶⁰ Integrin α9 inhibition with this antibody leads reduction into cytokine production in synovial macrophages in the mouse model of autoimmune arthritis.⁶¹ Since aging is the most common risk factor for both, acute ischemic stroke and VTE, we will utilize both, young (2-3-month-old) and aged (18-22 months-old mice) to test the efficacy of anti-integrin α9 integrin antibody. Young mice and aged WT mice with and without stroke will be treated with anti-integrin α9 antibody (55A2C) or control non-inhibitory antibody (18R18D). IVC stenosis will be induced 45 min post-treatment. Neutrophil adhesion at the stenosis site will be evaluated 4-hr post-stenosis as described previously in Aim 1a. The susceptibility to DVT (clot size, thrombosis incidence, clot properties) and tail-bleeding time will be evaluated as mentioned in Aim 1a.

To examine the effect of combining anti-integrin α9 antibody with current thromboprophylaxis regimen, in a separate set of mice with filament stroke model, we will inject low-molecular-weight heparin (15 mg/kg; enoxaparin) along with anti-integrin α9 antibody and will evaluate DVT outcomes as mentioned in Aim 1a.

Aim 2.2 Evaluate the effect of blocking integrin α9 in venous thrombosis using microfluidic device in human context: Human blood is neutrophil-rich (50-70% neutrophils, 30-50% lymphocytes) whereas mouse blood has a strong predominance of lymphocytes (75-90% lymphocytes, 10-25% neutrophils). Therefore, it is very important to evaluate translational feasibility of targeting integrin α9. To address this issue, we will utilize whole blood samples from the acute ischemic stroke patients. The magnitude of venous thrombosis will be evaluated using custom-made microfluidic device with dimensional similarity to human venous valves consists of a sinus distal to a sudden expansion⁶². Briefly, the flow chamber consists of 150 μm wide PDMS (polydimethylsiloxane) channels expanding into a 450 μm wide channels at angles 90°, 120°, 135°, or 150°, where larger angles represent a more severe undercut. The flow chamber will be coated with human umbilical vein endothelial cells (HUVECs) and will be activated with TNF-α (50 ng/ml), 3-hour before the experiment.

Study design: Under a currently approved protocol, we will collect blood samples from the patients with acute ischemic stroke and control blood samples from the age, sex, and other cardiovascular risk factors-matched patients. Blood sample will be collected within 6 hours of stroke patients receiving tPA or thrombectomy as this reflects our mouse stroke models. Since neutrophil numbers and functions are affected by infections, immunological disorders, and cancer, we will exclude such patients. Inclusion and exclusion criteria are shown in Table 3. Blood samples treated with anti-integrin α9 antibody, clone Y9A2 (5 μg/mL) or control antibody (anti-human IgG1 antibody clone AbD27686, 5 μg/mL) will be perfused in the microfluidic devise at the venous shear rate of 500 S-1 for 10 minutes. To evaluate venous thrombosis, platelets will be isolated from citrated whole blood, washed, labeled with the fluorescent dye (calcein AM; 2 μM), and reconstituted back with platelet-poor plasma and remaining blood containing leukocytes and red blood cells. Images will be acquired with an inverted microscope (Olympus IX81, ×20 numerical aperture [NA]=0.45, excitation/emission [ex/em] 475/505 nm) at 100 ms exposure. The fluorescently labeled platelets/thrombi on the endothelial cell coated surface will be analyzed using ImageJ software. We have prior experience with microfluidics flow chambers as evident by prior publications.^{63, 64}

TABLE 3
Enrollment Criteria

| Inclusions | Exclusions |
|---|---|
| 18-90 years of age | Pregnancy |
| Acute ischemic stroke | Hemorrhagic stroke |
| Treatment with rtPA/MT and blood | Signs of DVT/PE |
| collected within 6-hr | |
| of hospitalization | |
| | Signs of active infections |
| | Patients with immunological |
| | disorders |
| | Patients with active neoplasms |

Statistical analysis, sample Size & power considerations: Normally distributed data will be analyzed by Student's t-test or two-way ANOVA followed by Sidak's multiple comparisons test and non-normally distributed data will be analyzed using the Mann Whitney test (for two-group) or non-parametric two-way ANOVA followed by Fischer's LSD test. For mice studies, based on preliminary data, we estimate 25 mice per group to detect a significant difference between each group (>90% power, P<0.0125 with a Bonferroni adjustment for the four different groups). For studies using human samples, we will enroll 24 stroke patients and 24 respective controls per year (for 5 years) to have total sample size of 120 stroke patients and 120 controls. This sample size is sufficiently powered to analyze severity of in vitro venous thrombosis among groups. We will perform multivariable regression analysis to evaluate potential effect modifiers such as age, sex, stroke severity, comorbidities and reperfusion status. This is an exploratory basic science study to evaluate venous thrombosis following integrin α9 inhibition.

Without wishing to be bound by theory, treatment with anti-integrin α9 antibody will result into reduced post-stroke DVT in young mice with stroke as well as in aged mice. In microfluidic device study, without wishing to be bound theory, blood samples isolated from the stroke patients will display greater extent of venous thrombosis compared to control samples and targeting integrin α9 will reduce the extent of venous thrombosis. Such results can indicate that therapies targeting integrin α9 can be explored to reduce post-stroke VTE events. We have recently reported that targeting integrin α9 with a specific antibody 55A2C significantly improves stroke outcome in mouse models.³³ In case of no significant effect observed with single dose of antibody treatment in post-stroke DVT models, we will increase the dosing frequency and/or will employ small molecule inhibitors of integrin α9. In case of increased mortality in 18-22 months old mice, we will use 14-18 months old mice to evaluate efficacy of anti-integrin α9 integrin antibody in reducing DVT.

Mice will be randomized to treatment using a random number generator using Excel. Experiments are blinded to the experimental groups and treatments. For each surgery, before acquiring any data, the surgeon determines and records whether the surgery is technically successful, based on clear inclusion/exclusion criteria. We will include respective controls in each experiment and the data will be analyzed by a qualified statistician.

Completion of the experiments will indicate involvement of neutrophil integrin α9 in pathogenesis of DVT following stroke. In case of positive outcomes, we will test efficacy of targeting integrin α9 in thrombus resolution and with other comorbid conditions such as obesity/cancer, also in higher species such as rats. We will carry out pharmacokinetics, safety, and toxicological profiles of integrin α9 inhibitor that will allow for us to be ready for filing for new investigational drug application for the future clinical trials.

### References Cited in this Example

1. Silverstein M D, Heit J A, Mohr D N, Petterson T M, O'Fallon W M and Melton L J, 3rd. Trends in the incidence of deep vein thrombosis and pulmonary embolism: a 25-year population-based study. Arch Intern Med. 1998; 158:585-93.
2. Brandstater M E, Roth E J and Siebens H C. Venous thromboembolism in stroke: literature review and implications for clinical practice. Arch Phys Med Rehabil. 1992; 73: S379-91.
3. Kelly J, Rudd A, Lewis R R, Coshall C, Moody A and Hunt B J. Venous thromboembolism after acute ischemic stroke: a prospective study using magnetic resonance direct thrombus imaging. Stroke. 2004; 35:2320-5.
4. Kelly J, Rudd A, Lewis R and Hunt B J. Venous thromboembolism after acute stroke. Stroke. 2001; 32:262-7.
5. Rinde L B, Smabrekke B, Mathiesen E B, Lochen M L, Njolstad I, Hald E M, Wilsgaard T, Brackkan S K and Hansen J B. Ischemic Stroke and Risk of Venous Thromboembolism in the General Population: The Tromso Study. J Am Heart Assoc. 2016; 5.
6. Dennis M, Mordi N, Graham C, Sandercock P and collaboration Ct. The timing, extent, progression and regression of deep vein thrombosis in immobile stroke patients: observational data from the CLOTS multicenter randomized trials. Journal of thrombosis and haemostasis: JTH. 2011; 9:2193-200.
7. Sjalander A, Jansson J H, Bergqvist D, Eriksson H, Carlberg B and Svensson P. Efficacy and safety of anticoagulant prophylaxis to prevent venous thromboembolism in acutely ill medical inpatients: a metaanalysis. J Intern Med. 2008; 263:52-60.
8. Goldhaber S Z, Leizorovicz A, Kakkar A K, Haas S K, Merli G, Knabb R M, Weitz J I and Investigators A T. Apixaban versus enoxaparin for thromboprophylaxis in medically ill patients. N Engl J Med. 2011; 365:2167-77.
9. Sandercock P A, Counsell C and Kane E J. Anticoagulants for acute ischaemic stroke. Cochrane Database Syst Rev. 2015: CD000024.
10. The International Stroke Trial (IST): a randomised trial of aspirin, subcutaneous heparin, both, or neither among 19435 patients with acute ischaemic stroke. International Stroke Trial Collaborative Group. Lancet. 1997; 349:1569-81.
11. Diener H C, Ringelstein E B, von Kummer R, Landgraf H, Koppenhagen K, Harenberg J, Rektor I, Csanyi A, Schneider D, Klingelhofer J, Brom J, Weidinger G and Group P T. Prophylaxis of thrombotic and embolic events in acute ischemic stroke with the low-molecular-weight heparin certoparin: results of the PROTECT Trial. Stroke. 2006; 37:139-44.
12. Fuchs T A, Brill A, Duerschmied D, Schatzberg D, Monestier M, Myers D D, Jr., Wrobleski S K, Wakefield T W, Hartwig J H and Wagner D D. Extracellular DNA traps promote thrombosis. Proc Natl Acad Sci USA. 2010; 107:15880-5.
13. Brill A, Fuchs T A, Savchenko A S, Thomas G M, Martinod K, De Meyer S F, Bhandari A A and Wagner D D. Neutrophil extracellular traps promote deep vein thrombosis in mice. Journal of thrombosis and haemostasis: JTH. 2012; 10:136-44.
14. Fuchs T A, Brill A and Wagner D D. Neutrophil extracellular trap (NET) impact on deep vein thrombosis. Arteriosclerosis, thrombosis, and vascular biology. 2012; 32:1777-83.
15. von Bruhl M L, Stark K, Steinhart A, Chandraratne S, Konrad I, Lorenz M, Khandoga A, Tirniceriu A, Coletti R, Kollnberger M, Byrne R A, Laitinen I, Walch A, Brill A, Pfeiler S, Manukyan D, Braun S, Lange P, Riegger J, Ware J, Eckart A, Haidari S, Rudelius M, Schulz C, Echtler K, Brinkmann V, Schwaiger M, Preissner K T, Wagner D D, Mackman N, Engelmann B and Massberg S. Monocytes, neutrophils, and platelets cooperate to initiate and propagate venous thrombosis in mice in vivo. J Exp Med. 2012; 209:819-35.
16. Budnik I and Brill A. Immune Factors in Deep Vein Thrombosis Initiation. Trends Immunol. 2018; 39:610-623.
17. Schulz C, Engelmann B and Massberg S. Crossroads of coagulation and innate immunity: the case of deep vein thrombosis. Journal of thrombosis and haemostasis: JTH. 2013; 11 Suppl 1:233-41.
18. Stewart G J. Neutrophils and deep venous thrombosis. Haemostasis. 1993; 23 Suppl 1:127-40.
19. van Montfoort M L, Stephan F, Lauw M N, Hutten B A, Van Mierlo G J, Solati S, Middeldorp S, Meijers J C and Zeerleder S. Circulating nucleosomes and neutrophil activation as risk factors for deep vein thrombosis. Arteriosclerosis, thrombosis, and vascular biology. 2013; 33:147-51.
20. Rennard S I, Dale D C, Donohue J F, Kanniess F, Magnussen H, Sutherland E R, Watz H, Lu S, Stryszak P, Rosenberg E and Staudinger H. CXCR2 Antagonist MK-7123. A Phase 2 Proof-of-Concept Trial for Chronic Obstructive Pulmonary Disease. Am J Respir Crit Care Med. 2015; 191:1001-11.
21. Lazaar A L, Miller B E, Donald A C, Keeley T, Ambery C, Russell J, Watz H, Tal-Singer R and for I. CXCR2 antagonist for patients with chronic obstructive pulmonary disease with chronic mucus hypersecretion: a phase 2b trial. Respir Res. 2020; 21:149.
22. Dhanesha N, Nayak M K, Doddapattar P, Jain M, Flora G D, Kon S and Chauhan A K. Targeting myeloid-cell specific integrin alpha9beta1 inhibits arterial thrombosis in mice. Blood. 2020; 135:857-861.
23. Taooka Y, Chen J, Yednock T and Sheppard D. The integrin alpha9beta1 mediates adhesion to activated endothelial cells and transendothelial neutrophil migration through interaction with vascular cell adhesion molecule-1. The Journal of cell biology. 1999; 145:413-20.
24. Geerts W H, Pinco G F, Heit J A, Bergqvist D, Lassen M R, Colwell C W and Ray JG. Prevention of venous thromboembolism: the Seventh ACCP Conference on Antithrombotic and Thrombolytic Therapy. Chest. 2004; 126: 338S-400S.
25. McCarthy S T, Turner J J, Robertson D, Hawkey C J and Macey D J. Low-dose heparin as a prophylaxis against deep-vein thrombosis after acute stroke. Lancet. 1977; 2:800-1.
26. Courties G, Herisson F, Sager H B, Heidt T, Ye Y, Wei Y, Sun Y, Severe N, Dutta P, Scharff J, Scadden D T, Weissleder R, Swirski F K, Moskowitz M A and Nahrendorf M. Ischemic stroke activates hematopoietic bone marrow stem cells. Circ Res. 2015; 116:407-17.
27. Weisenburger-Lile D, Dong Y, Yger M, Weisenburger G, Polara G F, Chaigneau T, Ochoa R Z, Marro B, Lapergue B, Alamowitch S and Elbim C. Harmful neutrophil subsets in patients with ischemic stroke: Association with disease severity. Neurol Neuroimmunol Neuroinflamm. 2019; 6: e571.
28. Tang Y, Xu H, Du X, Lit L, Walker W, Lu A, Ran R, Gregg J P, Reilly M, Pancioli A, Khoury J C, Sauerbeck L R, Carrozzella J A, Spilker J, Clark J, Wagner K R, Jauch E C, Chang D J, Verro P, Broderick J P and Sharp F R. Gene expression in blood changes rapidly in neutrophils and monocytes after ischemic stroke in humans: a microarray study. J Cereb Blood Flow Metab. 2006; 26:1089-102.
29. Hoye A M, Couchman J R, Wewer U M, Fukami K and Yoneda A. The newcomer in the integrin family: integrin alpha9 in biology and cancer. Advances in biological regulation. 2012; 52:326-39.
30. Mambole A, Bigot S, Baruch D, Lesavre P and Halbwachs-Mecarelli L. Human neutrophil integrin alpha9beta1: up-regulation by cell activation and synergy with beta2 integrins during adhesion to endothelium under flow. J Leukoc Biol. 2010; 88:321-7.
31. Hallenbeck J M, Dutka A J, Tanishima T, Kochanek P M, Kumaroo K K, Thompson C B, Obrenovitch T P and Contreras T J. Polymorphonuclear leukocyte accumulation in brain regions with low blood flow during the early postischemic period. Stroke. 1986; 17:246-53.
32. Kataoka H, Kim S W and Plesnila N. Leukocyte-endothelium interactions during permanent focal cerebral ischemia in mice. J Cereb Blood Flow Metab. 2004; 24:668-76.
33. Dhanesha N, Jain M, Tripathi A, Doddapattar P, Chorawala M, Bathla G, Nayak M K, Ghatge M, Lentz S R, Kon S and Chauhan A K. Targeting Myeloid-Specific Integrin alpha9beta1 Improves Short and Long-Term Stroke Outcomes in Murine Models With Preexisting Comorbidities by Limiting Thrombosis And Inflammation. Circ Res. 2020; 126 (12) 1779-1794.
34. Chen C, Huang X, Atakilit A, Zhu Q S, Corey S J and Sheppard D. The Integrin alpha9beta1 contributes to granulopoiesis by enhancing granulocyte colony-stimulating factor receptor signaling. Immunity. 2006; 25:895-906.
35. Palmer E L, Ruegg C, Ferrando R, Pytela R and Sheppard D. Sequence and tissue distribution of the integrin alpha 9 subunit, a novel partner of beta 1 that is widely distributed in epithelia and muscle. The Journal of cell biology. 1993; 123:1289-97.
36. Takeuchi T, Tanaka Y, Erdman J, Kaneko Y, Saito M, Higashitani C, Smulders R and Lademacher C. ASP5094, a humanized monoclonal antibody against integrin alpha-9, did not show efficacy in patients with rheumatoid arthritis refractory to methotrexate: results from a phase 2a, randomized, double-blind, placebo controlled trial. Arthritis Res Ther. 2020; 22:252.
37. Dhanesha N, Schnell T, Rahmatalla S, DeShaw J, Thedens D, Parker B M, Zimmerman M B, Pieper A A, Chauhan A K and Leira E C. Low-Frequency Vibrations Enhance Thrombolytic Therapy and Improve Stroke Outcomes. Stroke. 2020; 51:1855-1861.
38. Dhanesha N, Vazquez-Rosa E, Cintron-Perez C J, Thedens D, Kort A J, Chuong V, Rivera-Dompenciel A M, Chauhan A K, Leira E C and Pieper A A. Treatment with Uric Acid Reduces Infarct and Improves Neurologic Function in Female Mice After Transient Cerebral Ischemia. J Stroke Cerebrovasc Dis. 2018; 27:1412-1416.
39. Dhanesha N, Ahmad A, Prakash P, Doddapattar P, Lentz S R and Chauhan A K. Genetic Ablation of Extra Domain A of Fibronectin in Hypercholesterolemic Mice Improves Stroke Outcome by Reducing Thrombo-Inflammation. Circulation. 2015; 132:2237-47.
40. Dhanesha N, Chorawala M R, Jain M, Bhalla A, Thedens D, Nayak M, Doddapattar P and Chauhan A K. Fn-EDA (Fibronectin Containing Extra Domain A) in the Plasma, but Not Endothelial Cells, Exacerbates Stroke Outcome by Promoting Thrombo-Inflammation. Stroke. 2019; 50:1201-1209.
41. Dhanesha N, Jain M, Doddapattar P, Undas A and Chauhan A K. Cellular fibronectin promotes deep vein thrombosis in diet-induced obese mice. Journal of thrombosis and haemostasis: JTH. 2021; 19:814-821.
42. Abram C L, Roberge G L, Hu Y and Lowell C A. Comparative analysis of the efficiency and specificity of myeloid-Cre deleting strains using ROSA-EYFP reporter mice. J Immunol Methods. 2014; 408:89-100.
43. Saha P, Andia M E, Modarai B, Blume U, Humphries J, Patel A S, Phinikaridou A, Evans C E, Mattock K, Grover S P, Ahmad A, Lyons O T, Attia R Q, Renne T, Premaratne S, Wicthoff A J, Botnar R M, Schaeffter T, Waltham M and Smith A. Magnetic resonance TI relaxation time of venous thrombus is determined by iron processing and predicts susceptibility to lysis. Circulation. 2013; 128:729-736.
44. Gupta S K and Vlahakis N E. Integrin alpha9beta1 mediates enhanced cell migration through nitric oxide synthase activity regulated by Src tyrosine kinase. J Cell Sci. 2009; 122:2043-54.
45. Lowell C A, Fumagalli L and Berton G. Deficiency of Src family kinases p59/61hck and p58c-fgr results in defective adhesion-dependent neutrophil functions. The Journal of cell biology. 1996; 133:895-910.
46. Abaricia J O, Shah A H and Olivares-Navarrete R. Substrate stiffness induces neutrophil extracellular trap (NET) formation through focal adhesion kinase activation. Biomaterials. 2021; 271:120715.
47. Nic M, Yang L, Bi X, Wang Y, Sun P, Yang H, Liu P, Li Z, Xia Y and Jiang W. Neutrophil Extracellular Traps Induced by IL8 Promote Diffuse Large B-cell Lymphoma Progression via the TLR9 Signaling. Clin Cancer Res. 2019; 25:1867-1879.
48. Nani S, Fumagalli L, Sinha U, Kamen L, Scapini P and Berton G. Src family kinases and Syk are required for neutrophil extracellular trap formation in response to beta-glucan particles. J Innate Immun. 2015; 7:59-73.
49. Campos J, Ponomaryov T, De Prendergast A, Whitworth K, Smith C W, Khan A O, Kavanagh D and Brill A. Neutrophil extracellular traps and inflammasomes cooperatively promote venous thrombosis in mice. Blood Adv. 2021; 5:2319-2324.
50. Roberts W G, Ung E, Whalen P, Cooper B, Hulford C, Autry C, Richter D, Emerson E, Lin J, Kath J, Coleman K, Yao L, Martinez-Alsina L, Lorenzen M, Berliner M, Luzzio M, Patel N, Schmitt E, LaGreca S, Jani J, Wessel M, Marr E, Griffor M and Vajdos F. Antitumor activity and pharmacology of a selective focal adhesion kinase inhibitor, PF-562,271. Cancer Res. 2008; 68:1935-44.
51. Paul R, Zhang Z G, Eliceiri B P, Jiang Q, Boccia A D, Zhang R L, Chopp M and Cheresh D A. Src deficiency or blockade of Src activity in mice provides cerebral protection following stroke. Nat Med. 2001; 7:222-7.
52. Prakash P, Kulkarni P P, Lentz S R and Chauhan A K. Cellular fibronectin containing extra domain A promotes arterial thrombosis in mice through platelet Toll-like receptor 4. Blood. 2015; 125:3164-72.
53. Chauhan A K, Kisucka J, Cozzi M R, Walsh M T, Moretti F A, Battiston M, Mazzucato M, De Marco L, Baralle F E, Wagner D D and Muro A F. Prothrombotic effects of fibronectin isoforms containing the EDA domain. Arteriosclerosis, thrombosis, and vascular biology. 2008; 28:296-301.
54. Fluri F, Schuhmann M K and Kleinschnitz C. Animal models of ischemic stroke and their application in clinical research. Drug Des Devel Ther. 2015; 9:3445-54.
55. Diaz J A, Saha P, Cooley B, Palmer O R, Grover S P, Mackman N, Wakefield T W, Henke P K, Smith A and Lal B K. Choosing a mouse model of venous thrombosis: a consensus assessment of utility and application. Journal of thrombosis and haemostasis: JTH. 2019; 17:699-707.
56. Diaz J A, Obi A T, Myers D D, Jr., Wrobleski S K, Henke P K, Mackman N and Wakefield T W. Critical review of mouse models of venous thrombosis. Arteriosclerosis, thrombosis, and vascular biology. 2012; 32:556-62.
57. Dhanesha N, Prakash P, Doddapattar P, Khanna I, Pollpeter M J, Nayak M K, Staber J M and Chauhan A K. Endothelial Cell-Derived von Willebrand Factor Is the Major Determinant That Mediates von Willebrand Factor-Dependent Acute Ischemic Stroke by Promoting Postischemic Thrombo-Inflammation. Arteriosclerosis, thrombosis, and vascular biology. 2016; 36:1829-37.
58. Schneider H, Harbottle R P, Yokosaki Y, Kunde J, Sheppard D and Coutelle C. A novel peptide, PLAEIDGIELTY, for the targeting of alpha9beta1-integrins. FEBS Lett. 1998; 429:269-73.
59. Liao Y F, Gotwals P J, Koteliansky V E, Sheppard D and Van De Water L. The EIIIA segment of fibronectin is a ligand for integrins alpha 9beta 1 and alpha 4beta 1 providing a novel mechanism for regulating cell adhesion by alternative splicing. The Journal of biological chemistry. 2002; 277:14467-74.
60. Kon S and Uede T. The role of alpha9beta1 integrin and its ligands in the development of autoimmune diseases. J Cell Commun Signal. 2018; 12:333-342.
61. Kanayama M, Kurotaki D, Morimoto J, Asano T, Matsui Y, Nakayama Y, Saito Y, Ito K, Kimura C, Iwasaki N, Suzuki K, Harada T, Li H M, Uchara J, Miyazaki T, Minami A, Kon S and Uede T. Alpha9 integrin and its ligands constitute critical joint microenvironments for development of autoimmune arthritis. J Immunol. 2009; 182:8015-25.
62. Lehmann M, Schoeman R M, Krohl P J, Wallbank A M, Samaniuk J R, Jandrot-Perrus M and Neeves K B. Platelets Drive Thrombus Propagation in a Hematocrit and Glycoprotein VI-Dependent Manner in an In Vitro Venous Thrombosis Model. Arteriosclerosis, thrombosis, and vascular biology. 2018; 38:1052-1062.
63. Nayak M K, Dhanesha N, Doddapattar P, Rodriguez O, Sonkar V K, Dayal S and Chauhan A K. Dichloroacetate, an inhibitor of pyruvate dehydrogenase kinases, inhibits platelet aggregation and arterial thrombosis. Blood Adv. 2018; 2:2029-2038.
64. Nayak M K, Ghatge M, Flora G D, Dhanesha N, Jain M, Markan K R, Potthoff M J, Lentz S R and Chauhan A K. The metabolic enzyme pyruvate kinase M2 regulates platelet function and arterial thrombosis. Blood. 2021; 137:1658-1668.

### Example 3

### Specific Aims:

Venous thromboembolism (VTE), including deep vein thrombosis (DVT) and pulmonary embolism (PE) have an immense impact on morbidity and mortality. Age is the most significant risk factor for developing VTE,¹ mainly due to the associated comorbiditics. Similarly, patients with stroke are also at high risk for developing life-threatening VTE events.²⁻⁶ While prophylactic anticoagulation reduces the rates of VTE in such high-risk patients, they only prevent approximately half of the VTE events.⁷ In addition, the use of anticoagulants is associated with significant risk of bleeding.⁷⁻¹¹ These data highlight the critical need for new and safe adjuvant treatments to reduce VTE burden in these patient population.

In recent years, compelling evidence has emerged that implicates neutrophils in the initiation and pathogenesis of deep vein thrombosis (DVT).¹²⁻¹⁹ Integrin activation is an essential step for neutrophil adhesion to the vasculature. Therapeutic targeting of neutrophil adhesion molecules such as CXCR2 are associated with neutropenia and increased incidence of infections.^{20, 21} Most adhesion molecules are expressed uniformly on the leukocytes and hence inhibition can affect innate immunity. In contrast, integrin α9 is highly expressed on neutrophils, expressed at low levels on monocytes, while the expression was not detected on lymphocytes and platelets.^{22, 23} We found that 1) stroke increases the risk of DVT, both in ice and humans. 2) integrin α9 is upregulated on neutrophils following stroke in humans and in mice and it contributes to adhesion of neutrophils to the endothelium, and 3) neutrophil specific α9−/− mice (α9fl/flMrp8Cre+/−) were less susceptible to DVT.

Despite the relevance of age and stroke in VTE, the effect of genetic deletion or pharmacological inhibition of integrin α9 on DVT severity in aged mice, as well as in mice with stroke, is not known. Additionally, the mechanisms by which neutrophil integrin α9 promotes DVT in the setting of stroke are not well established.

Neutrophils represent the majority of white blood cells in humans, but are less common in mice. It is not known whether inhibiting integrin α9 will reduce thrombosis in human blood at venous shear rate, in vitro. Also, it is not known whether inhibiting integrin α9 will reduce thrombosis in mice and humans with stroke treated with rtPA, a critical scenario where the use of prophylaxis anticoagulation is initially discouraged.

Without wishing to be bound by theory, neutrophil integrin α9 promotes DVT in the context of stroke and aging. The overall objective of the proposal is to validate the mechanisms by which neutrophil integrin α9 promotes DVT while exploring its relevance and therapeutic potential in reducing poststroke DVT. We will capitalize on our expertise in preclinical models of stroke (filament and embolic with rtPA), DVT (inferior vena cava stenosis) and in microfluidic device studies using blood samples from stroke patients to achieve following specific aims:

Aim 1: Determine the mechanistic role of neutrophil integrin α9 in promoting DVT in the context of stroke and aging. Our data clearly supports the role of neutrophil integrin α9 in promoting DVT. Without wishing to be bound by theory, integrin α9 promotes DVT in mice with stroke and in aged mice by producing neutrophil extracellular traps (NETs) and by promoting inflammasome activation. In Aim 1a, by utilizing neutrophil specific α9−/− mice with stroke, we will evaluate DVT in experimental models. In Aim 1b, we will utilize aged neutrophil specific α9−/− mice with and without stroke to evaluate DVT in the context of aging. In Aim 1c, using pharmacological inhibitors of the integrin α9 signaling pathway; we will evaluate whether integrin α9-FAK-SRC-Caspase-1 axis promotes NLRP3 inflammasome activation and NETosis. Using chimeric mice with stroke (α9fl/flMrp8Cre+/− bone-marrow transplanted in Fn-EDA+ mice), we will evaluate underlying mechanisms that contribute to neutrophil adhesion and DVT.

Aim 2: Determine the translational impact of inhibiting integrin α9 on venous thrombosis: We observed that inhibition of integrin α9 resulted into significant reduction in neutrophil adhesion to the activated endothelium. Without wishing to be bound by theory, integrin α9 inhibition will result into reduced venous thrombosis. In Aim 2a, we will utilize both, young and aged mice to test the in vivo effect of integrin α9 inhibition. Susceptibility to DVT after IVC stenosis in mice with and without stroke will be evaluated after integrin α9 inhibition in clinically relevant models and in absence or presence of thromboprophylaxis. In Aim 2b, utilizing whole blood samples from controls and patients with ischemic stroke, we will determine in vitro effect of integrin α9 inhibition on magnitude of venous thrombosis. For this, we will use endothelial cells coated, custom-made microfluidic device that mimics stenosed veins.

Outcome and Impact: Without wishing to be bound by theory, this project will provide robust evidence that neutrophil integrin α9 promotes DVT in the context of stroke and aging.

### References Cited in this Example

1. Silverstein M D, Heit J A, Mohr D N, Petterson T M, O'Fallon W M and Melton L J, 3rd. Trends in the incidence of deep vein thrombosis and pulmonary embolism: a 25-year population-based study. Arch Intern Med. 1998; 158:585-93.
2. Brandstater M E, Roth E J and Siebens H C. Venous thromboembolism in stroke: literature review and implications for clinical practice. Arch Phys Med Rehabil. 1992; 73: S379-91.
3. Kelly J, Rudd A, Lewis R R, Coshall C, Moody A and Hunt B J. Venous thromboembolism after acute ischemic stroke: a prospective study using magnetic resonance direct thrombus imaging. Stroke. 2004; 35:2320-5.
4. Kelly J, Rudd A, Lewis R and Hunt B J. Venous thromboembolism after acute stroke. Stroke. 2001; 32:262-7.
5. Rinde L B, Smabrekke B, Mathiesen E B, Lochen M L, Njolstad I, Hald E M, Wilsgaard T, Brackkan S K and Hansen J B. Ischemic Stroke and Risk of Venous Thromboembolism in the General Population: The Tromso Study. J Am Heart Assoc. 2016; 5.
6. Dennis M, Mordi N, Graham C, Sandercock P and collaboration Ct. The timing, extent, progression and regression of deep vein thrombosis in immobile stroke patients: observational data from the CLOTS multicenter randomized trials. Journal of thrombosis and haemostasis: JTH. 2011; 9:2193-200.
7. Sjalander A, Jansson J H, Bergqvist D, Eriksson H, Carlberg B and Svensson P. Efficacy and safety of anticoagulant prophylaxis to prevent venous thromboembolism in acutely ill medical inpatients: a metaanalysis. J Intern Med. 2008; 263:52-60.
8. Goldhaber S Z, Leizorovicz A, Kakkar A K, Haas S K, Merli G, Knabb R M, Weitz J I and Investigators A T. Apixaban versus enoxaparin for thromboprophylaxis in medically ill patients. N Engl J Med. 2011; 365:2167-77.
9. Sandercock P A, Counsell C and Kane E J. Anticoagulants for acute ischaemic stroke. Cochrane Database Syst Rev. 2015: CD000024.
10. The International Stroke Trial (IST): a randomised trial of aspirin, subcutaneous heparin, both, or neither among 19435 patients with acute ischaemic stroke. International Stroke Trial Collaborative Group. Lancet. 1997; 349:1569-81.
11. Diener H C, Ringelstein E B, von Kummer R, Landgraf H, Koppenhagen K, Harenberg J, Rektor I, Csanyi A, Schneider D, Klingelhofer J, Brom J, Weidinger G and Group P T. Prophylaxis of thrombotic and embolic events in acute ischemic stroke with the low-molecular-weight heparin certoparin: results of the PROTECT Trial. Stroke. 2006; 37:139-44.
12. Fuchs T A, Brill A, Duerschmied D, Schatzberg D, Monestier M, Myers D D, Jr., Wrobleski S K, Wakefield T W, Hartwig J H and Wagner D D. Extracellular DNA traps promote thrombosis. Proc Natl Acad Sci USA. 2010; 107:15880-5.
13. Brill A, Fuchs T A, Savchenko A S, Thomas G M, Martinod K, De Meyer S F, Bhandari A A and Wagner D D. Neutrophil extracellular traps promote deep vein thrombosis in mice. Journal of thrombosis and haemostasis: JTH. 2012; 10:136-44.
14. Fuchs T A, Brill A and Wagner D D. Neutrophil extracellular trap (NET) impact on deep vein thrombosis. Arteriosclerosis, thrombosis, and vascular biology. 2012; 32:1777-83.
15. von Bruhl M L, Stark K, Steinhart A, Chandraratne S, Konrad I, Lorenz M, Khandoga A, Tirniceriu A, Coletti R, Kollnberger M, Byrne R A, Laitinen I, Walch A, Brill A, Pfeiler S, Manukyan D, Braun S, Lange P, Riegger J, Ware J, Eckart A, Haidari S, Rudelius M, Schulz C, Echtler K, Brinkmann V, Schwaiger M, Preissner K T, Wagner D D, Mackman N, Engelmann B and Massberg S. Monocytes, neutrophils, and platelets cooperate to initiate and propagate venous thrombosis in mice in vivo. J Exp Med. 2012; 209:819-35.
16. Budnik I and Brill A. Immune Factors in Deep Vein Thrombosis Initiation. Trends Immunol. 2018; 39:610-623.
17. Schulz C, Engelmann B and Massberg S. Crossroads of coagulation and innate immunity: the case of deep vein thrombosis. Journal of thrombosis and haemostasis: JTH. 2013; 11 Suppl 1:233-41.
18. Stewart G J. Neutrophils and deep venous thrombosis. Haemostasis. 1993; 23 Suppl 1:127-40.
19. van Montfoort M L, Stephan F, Lauw M N, Hutten B A, Van Mierlo G J, Solati S, Middeldorp S, Meijers J C and Zeerleder S. Circulating nucleosomes and neutrophil activation as risk factors for deep vein thrombosis. Arteriosclerosis, thrombosis, and vascular biology. 2013; 33:147-51.
20. Rennard S I, Dale D C, Donohue J F, Kanniess F, Magnussen H, Sutherland E R, Watz H, Lu S, Stryszak P, Rosenberg E and Staudinger H. CXCR2 Antagonist MK-7123. A Phase 2 Proof-of-Concept Trial for Chronic Obstructive Pulmonary Disease. Am J Respir Crit Care Med. 2015; 191:1001-11.
21. Lazaar A L, Miller B E, Donald A C, Keeley T, Ambery C, Russell J, Watz H, Tal-Singer R and for I. CXCR2 antagonist for patients with chronic obstructive pulmonary disease with chronic mucus hypersecretion: a phase 2b trial. Respir Res. 2020; 21:149.
22. Dhanesha N, Nayak M K, Doddapattar P, Jain M, Flora G D, Kon S and Chauhan A K. Targeting myeloid-cell specific integrin alpha9beta1 inhibits arterial thrombosis in mice. Blood. 2020; 135:857-861.
23. Taooka Y, Chen J, Yednock T and Sheppard D. The integrin alpha9beta1 mediates adhesion to activated endothelial cells and transendothelial neutrophil migration through interaction with vascular cell adhesion molecule-1. The Journal of cell biology. 1999; 145:413-20.

### Example 4

### Protocol to Study the Effect of Macitentan in Lung-Cancer Model.

### Cell Lines

Lewis lung carcinoma (LLC1) cell line will be obtained from American Type Culture Collection (ATCC). The cell line was originally derived from a Lewis lung carcinoma of a C57BL mouse strain. The cells will be grown in Dulbecco's Modified Eagle Medium (DMEM) supplemented with 10% FCS, 2 mM L-glutamine, 100 U/ml penicillin, 100 μg/ml streptomycin, and 0.1% fungizone. The cells will be maintained at 37° C. in a humidified atmosphere with 5% CO2. The LLC1 cells (100 μL) will be injected once intravenously.

### Macitentan Treatment

Macitentan at the dose of 2, 5 and 10 mg/kg will be administered orally once a day from the day of cancer induction until the end of the study.

### Venous Thrombosis:

Deep vein thrombosis (DVT) will be induced in mice after 14 days of tumor induction. Briefly, anesthetized mice will undergo laparotomy, the intestine will be exteriorized, and the intra renal IVC will be exposed. The IVC will then be dissected away from the aorta. For stenosis, IVC will be exposed, a space holder (30-gauge, 3-mm long needle) will be positioned on the outside of the vessel, and a permanent narrowing ligature will be placed exactly below the left renal vein. Subsequently, the needle will be removed to restrict blood flow to 80-90%. Throughout the surgery, the body temperature of the mice will be maintained at 37° C.±1.0 using a heating pad. Buprenorphine (0.1 mg/kg, SC) will be administered as an analgesic agent at every 6-12 hours for 48 hours post-surgery.

### Outcome Measures.

Thrombus size and thrombosis incidence on day 1 and day 2 following DVT surgery will be evaluated by ultrasonography.

### Example 5

### Neutrophil Integrin α9β1 and Venous Thrombosis

Venous thromboembolism (VTE) encompasses deep-vein thrombosis (DVT) and pulmonary embolism. VTE is the leading cause of lost disability-adjusted life years and is the third leading cause of cardiovascular death in the world. Identification and validation of a therapeutic target that limits thrombosis and inflammation without causing major bleeding, holds a significant promise to reduce VTE burden.

### Integrins and Neutrophil Adhesion

Initial adhesion and trans-endothelial migration of neutrophils depends on expression and upregulation of various adhesion molecules including integrins. Integrin α9β1 is expressed on neutrophils, upregulated upon activation, stabilizes adhesion to activated endothelium, and plays a significant role in transmigration. Integrin alpha-9 binds to many ligands such as VCAM-1, ICAM-1,TSP-1 and extracellular matrix protein including Fibronectin.

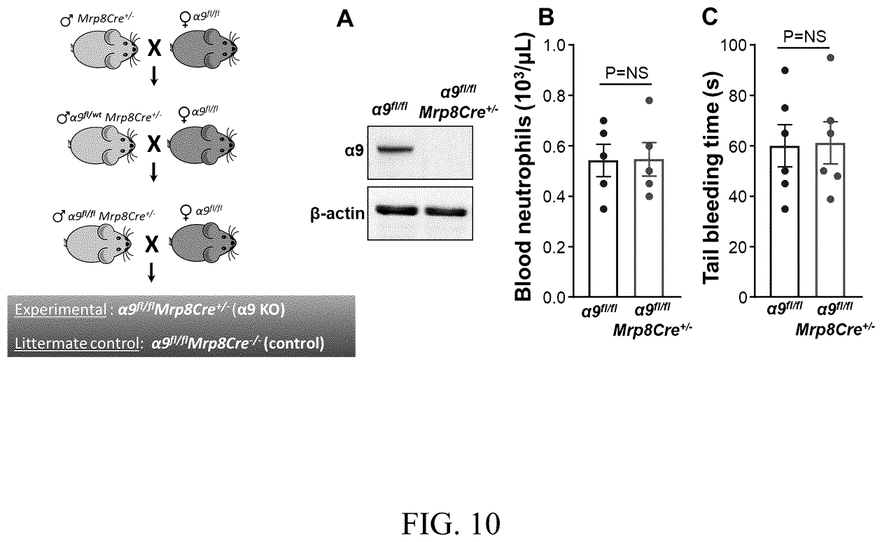

Referring to FIG. 10, data relating to neutrophil specific integrin alpha-9 deficient mice is provided.

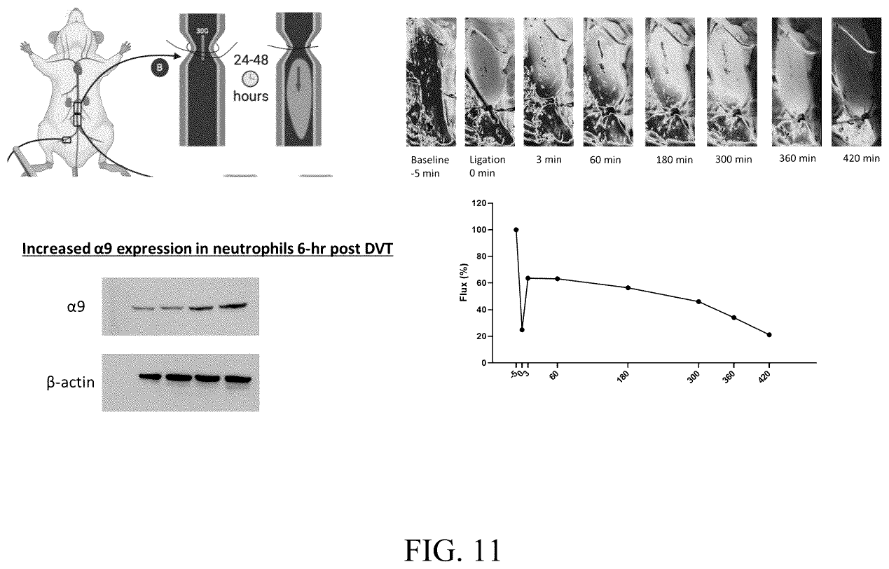

Referring to FIG. 11, data showing increased α9 expression in neutrophils six hours post DVT in an IVC stenosis mouse model is provided.

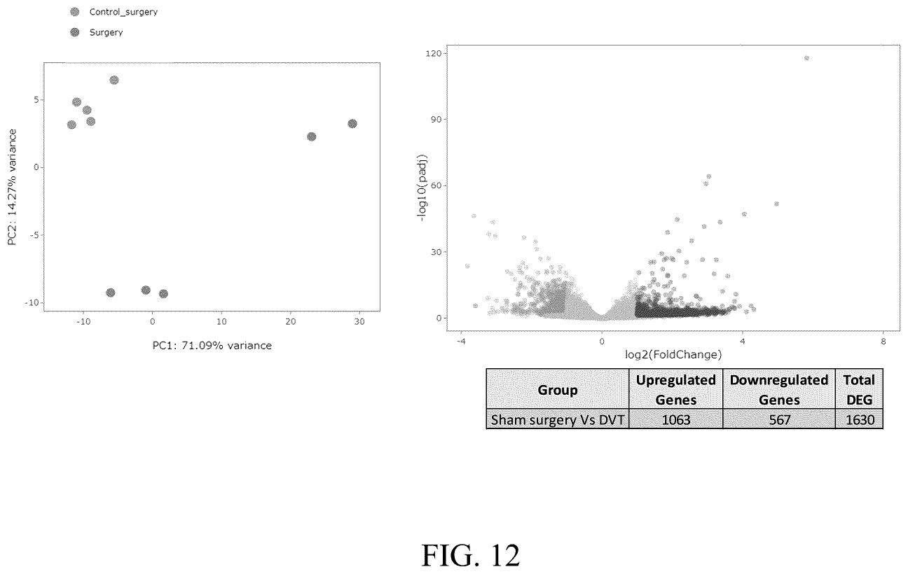

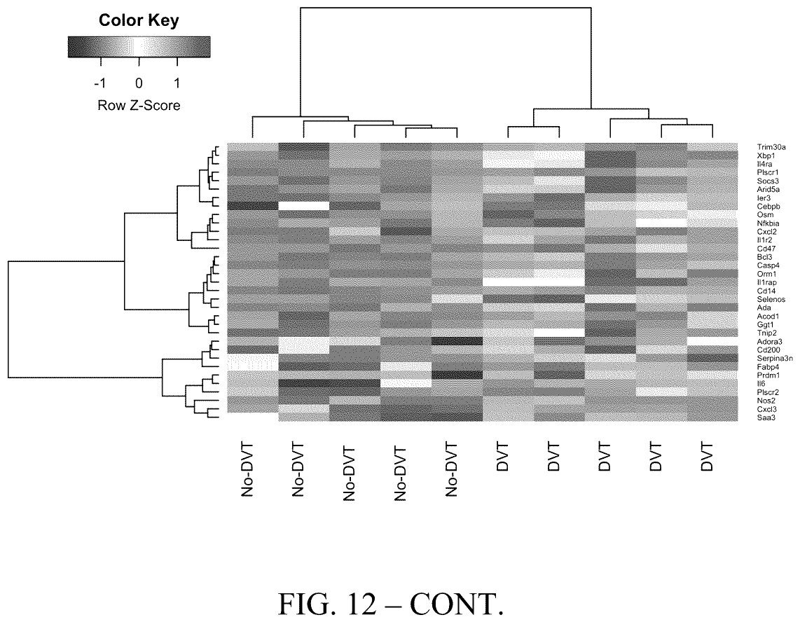

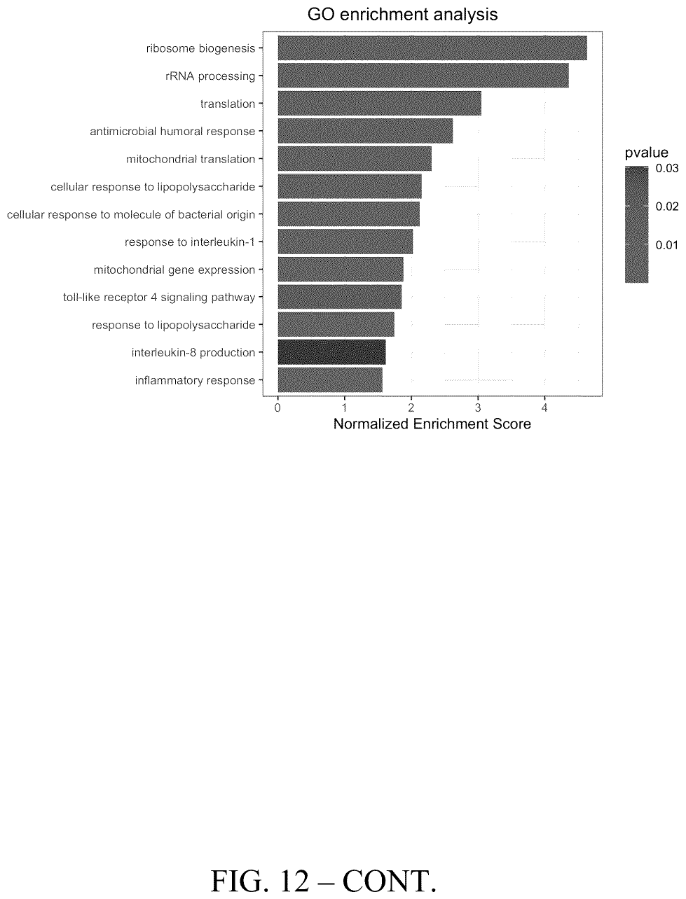

Referring to FIG. 12, data showing differentially regulated genes in neutrophils of control and mice with DVT is provided.

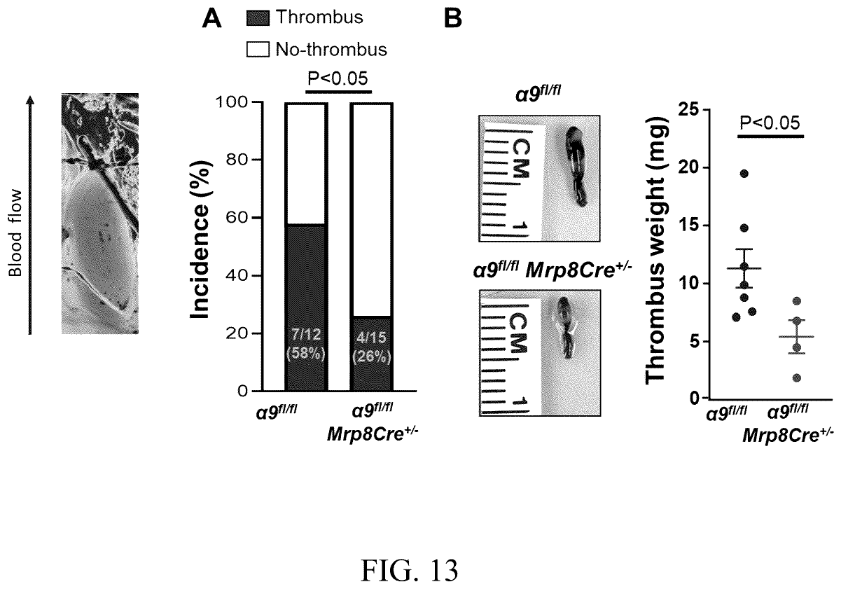

Referring to FIG. 13, data showing that neutrophil-specific α9 mice exhibit reduced DVT severity is provided. DVT outcome was evaluated using IVC stenosis in neutrophil-specific α9^{-/-} mice. Significantly reduced DVT incidence and thrombus weight in neutrophil-specific α9−/− mice was observed.

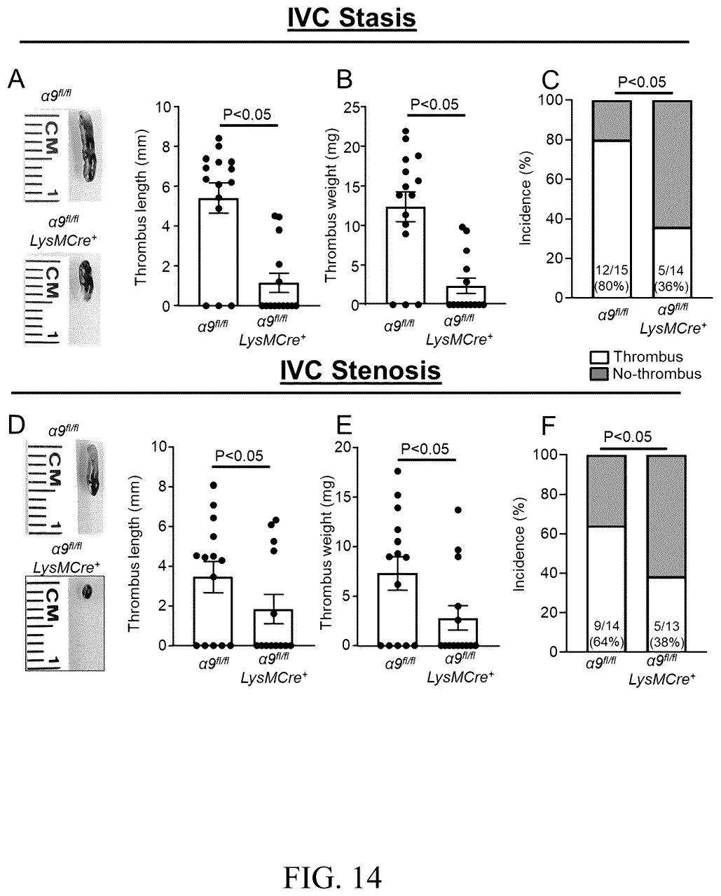

Referring to FIG. 14, data showing that myeloid cell-specific α9 mice exhibit reduced DVT severity in IVC stasis (Panel A) and stenosis (Panel B) models is provided.

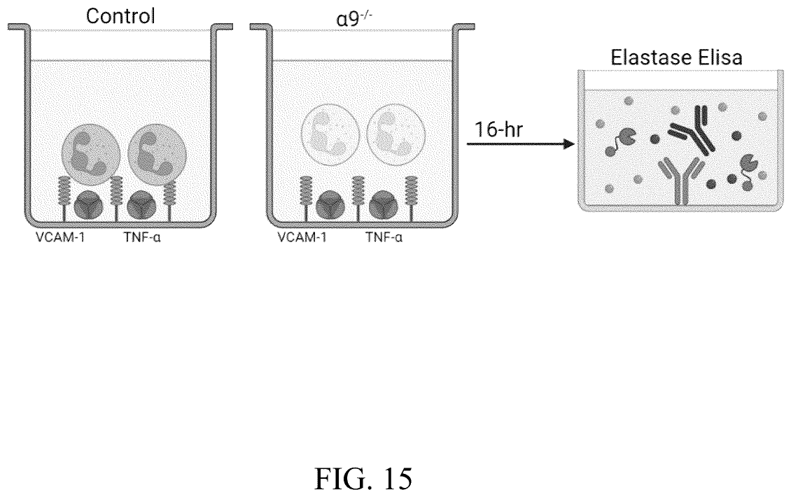

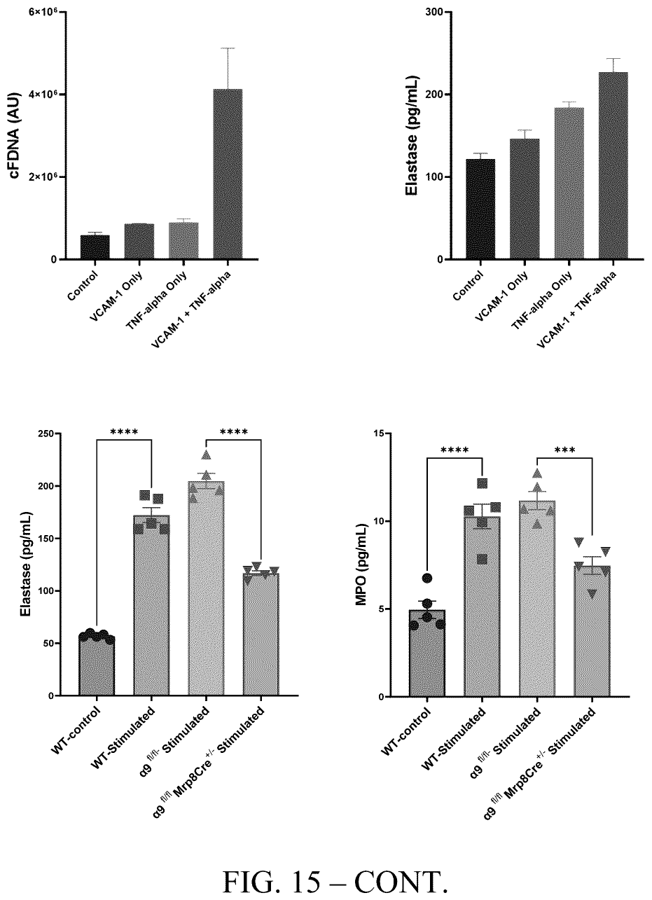

Referring to FIG. 15, data relating to α9-VCAM1 interactions is provided.

### Homology Model Building

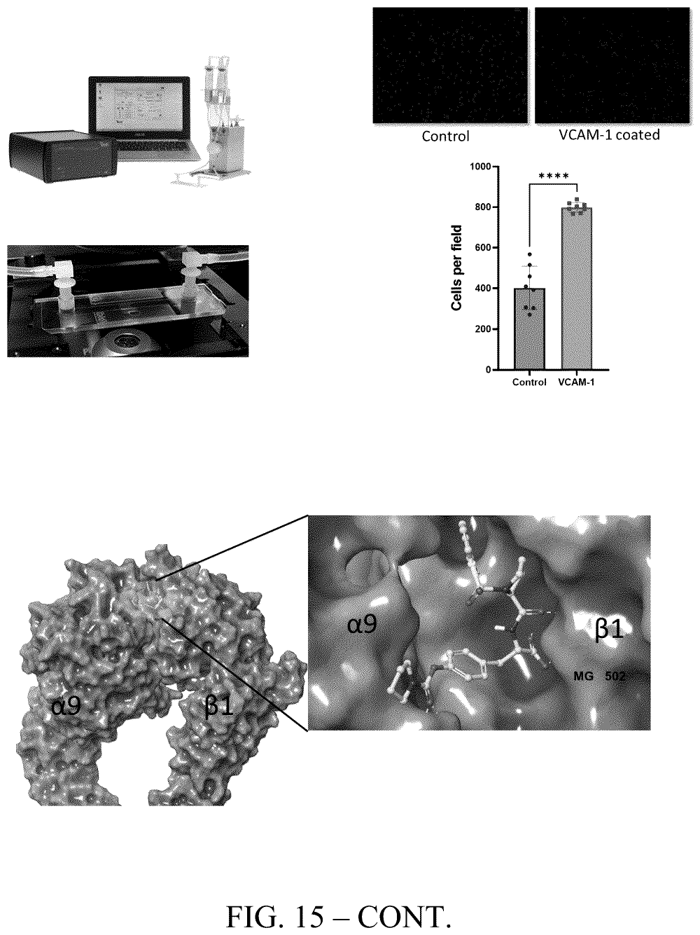

Referring to FIG. 16, a flow chart showing homology model building is provided. Integrin α9 Sequence was retrieved from uniport (ID: Q13797). Integrin α4 (PDB ID: 3V4P, α4β1-hetrodimer) unit was used as template protein for model building. β1 subunit (from α4β1-hetrodimer) was merged with the α9 integrin, to form α9β1. Ca²⁺ and Mg²⁺ were retained from the α4β1 complex, as they impart stability to the protein and help in the ligand binding. The built model was energetically optimized to remove the steric clashes and was validated.

### Ligand Database Preparation

FDA approved drugs were downloaded from Selleckchem database. Only small molecules were used to prepare the internal database. Large molecules (Biologics) were removed. Duplicates molecules were removed. The cleaned 2D molecules were converted to 3D using OpenBabel 2.3.2.

### Docking Steps (PyrX 0.9v)

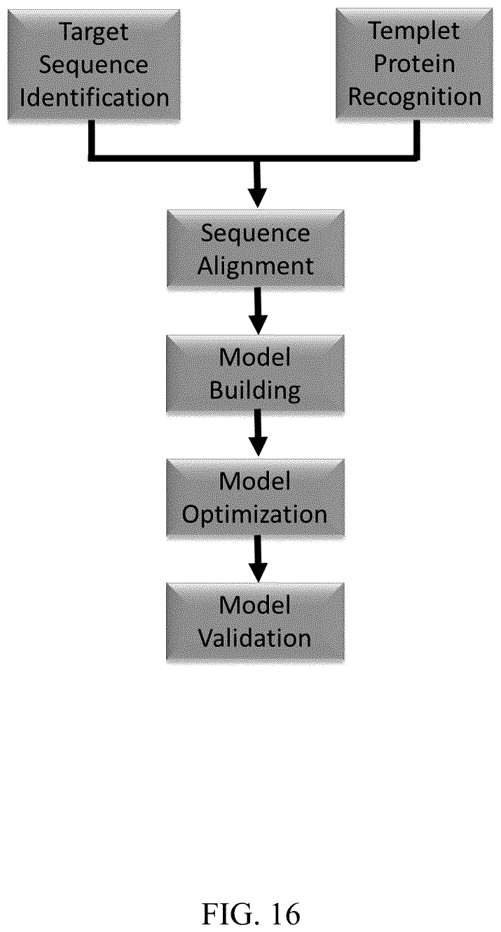

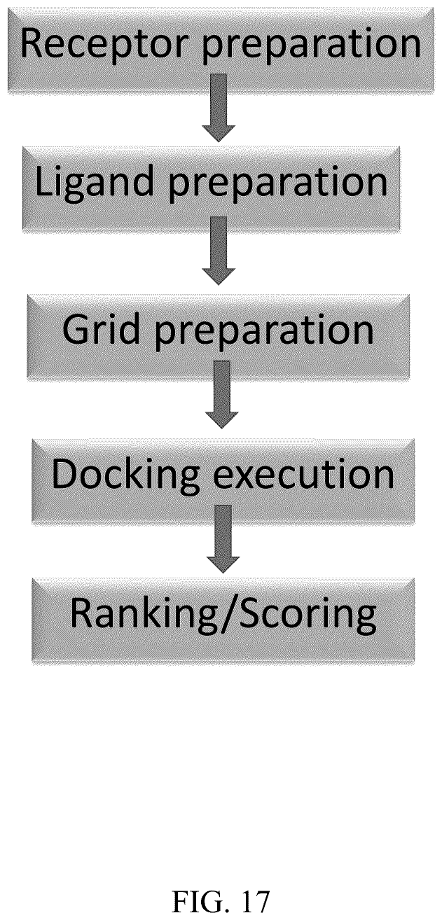

Referring to FIG. 17, a flow chart showing the docking steps is provided. Receptor preparation: Built model was used to dock the ligands. Hydrogens and charges were added, missing side chains were filled and converted to PDBQT. Ligand preparation: Ligands were converted to PDBQT format and optimized. Grid generation: Binding/active site residues were selected. Docking execution: Compounds were allowed to fit into the receptor. Ranking/scoring: Hits were ranked based on the docking score/number of interactions.

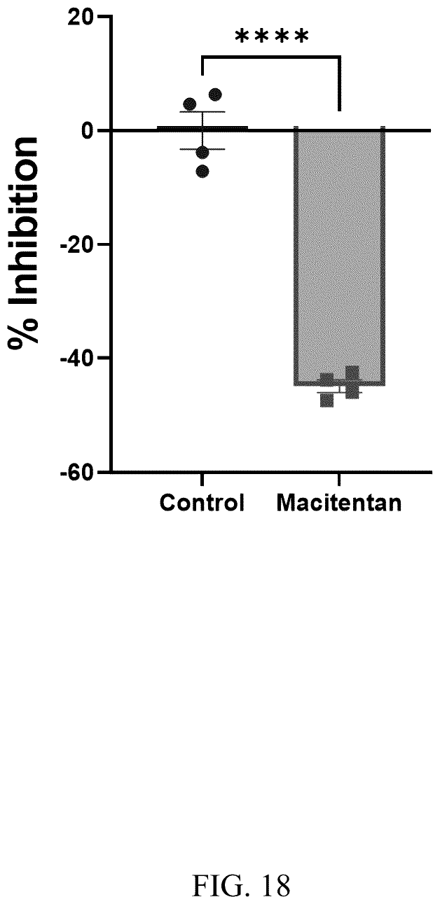

Referring to FIG. 18, a bar graph showing % inhibition of alpha-9 VCAM-1 binding by Macitentan is provided.

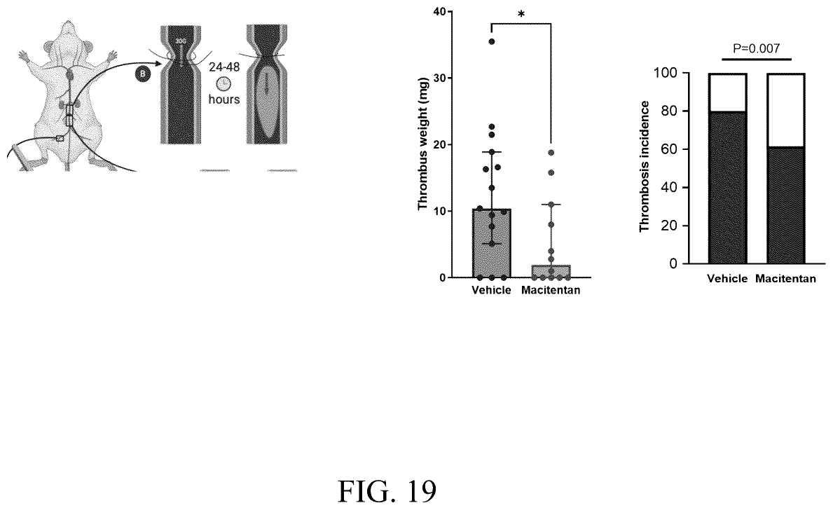

Referring to FIG. 19, data showing in vivo efficacy of Macitentan in WT mice is provided.

### Example 6

### Methods of Preventing a Venous Thromboembolic Event

Cancer associated thrombosis is highly prevalent with limited treatment options. The incidence of VTE in general population is relatively low, (˜1 per 1000 per year), while among cancer patients, the occurrence of VTE is 3-10 times higher, depending on the type and the stage of cancer. Primary thromboprophylaxis with low-molecular-weight heparin (LMWH) or with Factor Xa inhibitors reduces thrombosis incidence, but is associated with significantly increased risk of bleeding without improving overall survival for patients with cancer.

Despite major advances in our understanding of the mechanisms that lead to cancer associated thrombosis and considerable efforts in drug development, no pharmacological therapy currently exists to reduce cancer associated thrombosis without bleeding side effects. Thus, there is an unmet need for new interventions that synergize with current thromboprophylaxis regimens to reduce the rate of VTE in such high-risk patients.

The technology described herein has significant novelty for the following reasons:
1) Utilizing a new mouse model of cancer associated thrombosis has demonstrated that Macitentan treatment resulted in significant reduction in severity of deep vein thrombosis;
2) Without wishing to be bound by theory, this will be a first-in-class therapy for the prevention of venous thrombosis in patients with cancer devoid of any bleeding side effects;
3) It is shown that Macitentan binds to integrin α9 and inhibits its interactions with VCAM-1; and
4) It is shown that there is significant expression of neutrophil integrin α9 in human lung cancer tissue, implicating the functional relevance for the clinical translation.

In a study examining the association of resource utilization and real-world costs in ambulatory patients initiating chemotherapy for selected common high-risk solid tumors, it was found that cancer patients who experienced venous thrombosis had approximately three times as many all-cause hospitalizations (mean 1.38 versus 0.55 per patient; P<0.0001) and days in hospital (mean 10.19 versus 3.37 per patient; P<0.0001) as cancer patients without venous thrombosis. Furthermore, outpatient medical claims (mean 291.44 versus 173.39 per patient; P<0.0001) and outpatient prescription claims (mean 39.97 versus 33.07 per patient; P<0.0001) were also higher in cancer patients with venous thrombosis versus those without venous thrombosis. Cancer patients who experienced venous thrombosis also incurred significantly higher (unadjusted) overall (all-cause) inpatient costs (mean USD 21,299 versus USD 7459 per patient; P<0.0001), outpatient medical costs (mean USD 47,091 versus USD 29,901 per patient; P<0.0001), outpatient prescription costs (mean USD 6569 versus USD 4331 per patient; P<0.0001) and total health care costs (mean USD 74,959 versus USD 41,691 per patient; P<0.0001) over the 12-month post-thrombosis follow-up period than those without venous thrombosis.

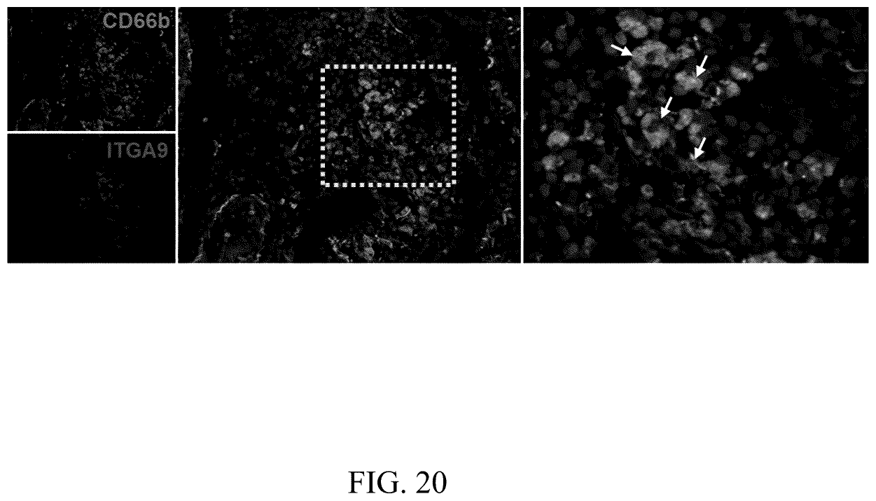

Referring to FIG. 20, significant expression of neutrophil integrin α9 levels in histopathologically diagnosed non-small cell lung cancer cases is shown. Neutrophils (CD66b+; green); Integrin α9 (ITGA9; red); (n=8).

### Results

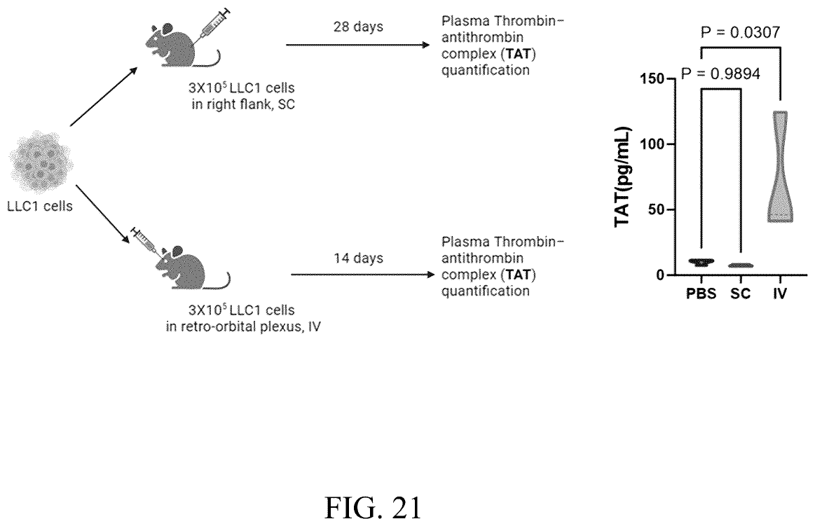

Referring to FIG. 21, a schematic showing an optimized, new murine model of lung cancer which exhibits enhanced coagulopathy is shown. Male WT mice were injected with LLC1 cells, IV or SC. The markers of coagulations were increased in mice with IV injection of LLC1 cells.

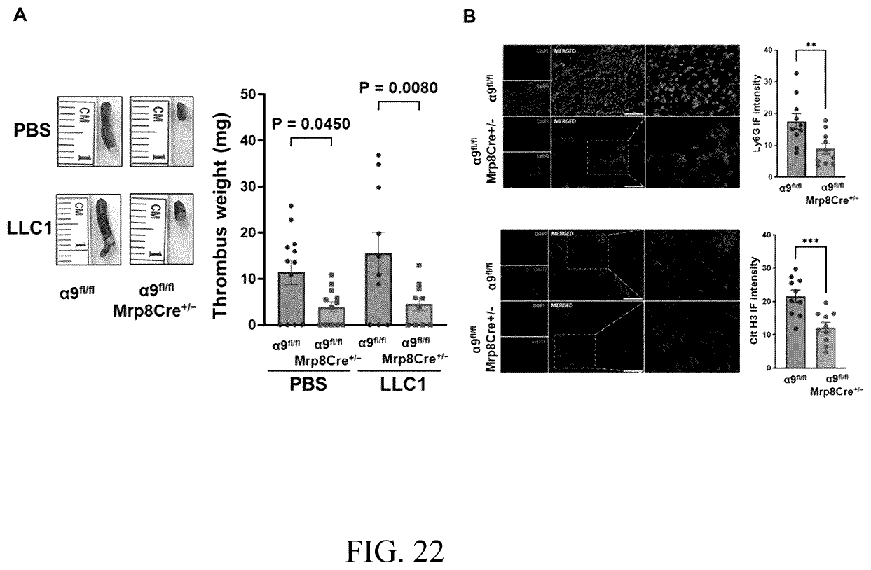

Referring to FIG. 22, data showing neutrophil specific α9 deficient mice exhibit significantly reduced DVT severity in mice with lung-cancer is provided. (Panel A) Left, representative IVC thrombus harvested 48-hour post-stenosis from each group. Right, thrombus weight (mg). Each dot represents a single mouse. (Panel B) Left, representative cross-sectional immunofluorescence image of the isolated IVC thrombus (48-hour post-stenosis) from each group for Ly6G (neutrophils, green), CitH3 (red), and DAPI (blue). Magnification 20×; Scale bar 50 μm. Right, quantification.

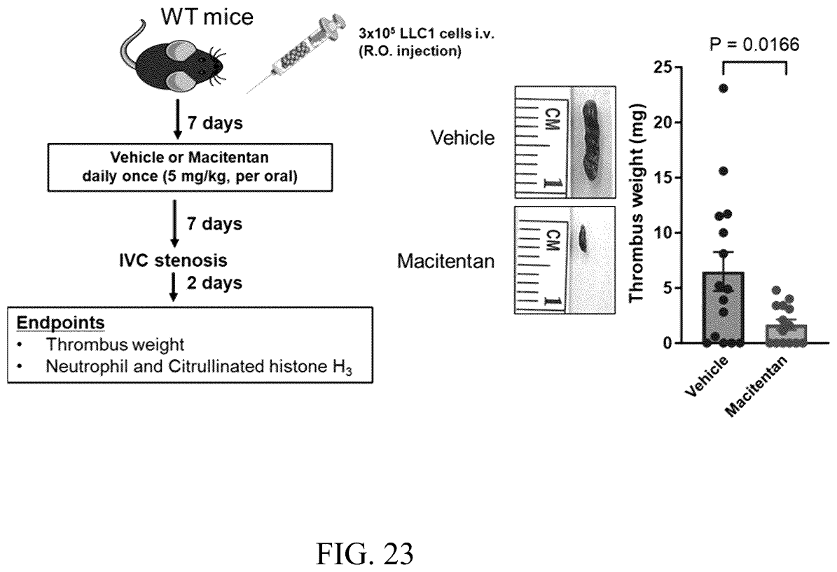

Referring to FIG. 23, data showing that Macitentan treatment significantly reduced DVT severity in mice with lung cancer is provided. Panel A provides a schematic showing the experimental design. Panel B provides data showing representative IVC thrombus harvested 48-hour post-stenosis from each group (Left), and thrombus weight (mg) (Right). Each dot represents a single mouse.

### Example 7

### Macitentan for the Prevention of Cancer Associated Thrombosis (M-CAT)

### Study Description

Venous thromboembolism (VTE), including deep vein thrombosis (DVT) and pulmonary embolism (PE), have immense impact on morbidity and mortality. Patients with cancer have a 7 to 28-fold higher risk for venous thromboembolism (VTE) than non-cancer patients. Patients with brain, pancreatic, stomach, lung cancers, and patients who are receiving chemotherapy or immunotherapy, are at the highest risk for developing blood clots. Occurrence of VTE in cancer patients can result in reduced life expectancy, delayed cancer treatment, and overall reduced quality of life. While prophylactic anticoagulation can reduce the rates of blood clots, they only prevent approximately half of the events and are associated with significant risk of bleeding, suggesting the critical need for new and safe adjuvant treatments to reduce blood clots in high-risk patients.

Using animal models and several cell-based assays, we recently discovered that Macitentan reduces incidence of DVT. Macitentan is an endothelin receptor antagonist and approved by the FDA for the treatment of pulmonary arterial hypertension. In the current study, we will first identify cancer patients who are at a high risk of developing VTE by using a validated tool at the time of their cancer diagnosis. These high-risk patients will be asked to participate in a trial to test the safety and efficacy of Macitentan. In this pilot and feasibility study, we are proposing to enroll 50 patients. These patients will be randomized to receive Macitentan (n=25) or placebo (n=25). Analysis will be performed to assess the safety (rate of clinical overt bleeding (major and minor bleeding) and death within the study period) and the efficacy [first episode of objectively documented, symptomatic or asymptomatic VTE (DVT and/or PE)] of Macitentan.

### Inclusion Criteria

Inclusion criteria for the study includes the following:
Age 18 years old or older.
Provide written informed consent.
Have histologically confirmed solid malignancy (locally advanced or metastatic disease) including, but not limited to, brain cancer, pancreatic cancer, stomach cancer, lung cancer, colorectal cancer, bladder cancer, ovarian cancer, and/or renal cancer
Hematological malignancies such as lymphomas and multiple myeloma
Have an Eastern Cooperative Oncology Group (ECOG) Performance Status of 0-2
Elevated D-Dimer (≥1.44 μg/mL)
Plan to initiate systemic cancer therapy (chemotherapy and/or immunotherapy) within plus or minus (+/−) 1 week of receiving the first dose of study drug with the intention of receiving systemic cancer therapy during the study period for an intended duration determined by the treating oncologist according to standard protocols of clinical care

### Exclusion Criteria

Exclusion criteria for the study includes the following:
Lesions or conditions at increased risk of clinically significant bleeding (e.g. active peptic ulcer disease)
Objectively confirmed substantial liver insufficiency as defined by clinical manifestations of ascites, cirrhosis, encephalopathy and/or jaundice and/or biochemical abnormalities in liver function tests including hypoalbuminemia (<3.5 gr/dL), elevated levels of total bilirubin (>25 μmol/L), elevated liver transaminases (2 times the upper limit of normal) and/or biochemical diagnosis of biliary tract obstruction (elevated levels of gamma-glutamyl transferase and alkaline phosphatase, 3 times the upper limit of normal)
Planned stem cell transplant
Life expectancy less than 6 months
Pregnancy
Continuous anticoagulation with vitamin K antagonists, low-molecular-weight heparin (LMWH), or other oral anticoagulants.
Weight <40 Kg
Known allergies to ingredients contained in Macitentan
Use of any contraindicated medications with Macitentan

### Arms and Interventions

| Participant Group/Arm | Intervention/Treatment |
|---|---|
| Active Comparator: Macitentan | Drug: Macitentan |
| 10 mg daily once | Macitentan: 10 mg |
| for 6 months | daily once for 6 months |
| | Other Names: |
| | OPSUMIT ® |
| Placebo Comparator: Placebo drug | Drug: Placebo drug |
| 10 mg daily | 10 mg daily |
| once for 6 months | once for 6 months |

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method of preventing, treating, or alleviating a symptom of a venous thromboembolic event in a subject, the method comprising administering to the subject in need thereof an endothelin receptor inhibitor.

2. The method of claim 1, wherein the endothelin receptor inhibitor comprises an integrin alpha9 (ITGA9) inhibitor.

3. The method of claim 2, wherein the ITGA9 inhibitor comprises a small molecule, an antibody or fragment thereof, or a nucleic acid.

4. The method of claim 3, wherein the small molecule comprises a structure according to the following, or a derivative or analog thereof:

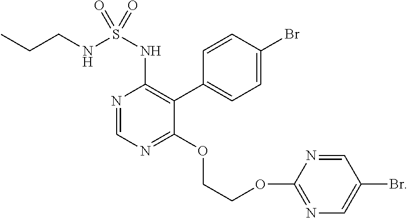 [SMILES: CCCNS(=O)(=O)Nc1ncnc(OCCOc2ncc(Br)cn2)c1-c1ccc(Br)cc1]

5. The method of claim 1, wherein the venous thromboembolic event comprises venous thrombosis, pulmonary embolism, or cancer-associated thrombosis.

6. The method of claim 5, wherein venous thrombosis comprises deep vein thrombosis.

7. The method of claim 1, wherein the subject is afflicted with a cancer.

8. The method of claim 7, wherein the cancer comprises a solid tumor cancer.

9. The method of claim 8, wherein the solid tumor cancer comprises lung cancer, brain cancer, stomach cancer, or pancreatic cancer.

10. The method of claim 1, wherein the subject is or has been administered a therapeutic regimen associated with increased incidence of a venous thromboembolic event.

11. The method of claim 10, wherein the therapeutic regimen comprises an anti-cancer therapeutic regimen.

12. The method of claim 11, wherein the anti-cancer therapeutic regimen comprises chemotherapy, radiotherapy, immunotherapy, surgery, or any combination thereof.

13. The method of claim 1, further comprising administering to the subject a thromboprophylaxis regimen.

14. The method of claim 13, wherein the thromboprophylaxis regimen comprises an antithrombotic agent.

15. The method of claim 14, wherein the antithrombotic agent comprises warfarin, heparin, dabigatran, rivaroxaban, or apixaban.
